(19) United States
(12) Patent Application Publication
(10) Pub. No.: US 2004/0072259 A1
(43) Pub. Date: Apr. 15, 2004

(54) Methods and products for manipulating hematopoietic stem cells

(76) Inventors: David T. Scadden, Weston, MA (US); Byeong-Chel Lee, Cambridge, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/433,146

(22) PCT Filed: Nov. 29, 2001
(86) PCT No.: PCT/US01/45076

(51) Int. Cl. C12Q 1/68; G01N 33/53; G01N 33/567

(52) U.S. Cl. 435/7.2; 435/6

(57) ABSTRACT

The invention relates to methods for manipulating hematopoietic stem cells and related products. In one aspect the invention relates to the use of stem cell G-protein coupled receptor (SC-GPR) related compositions to identify bone marrow derived hematopoietic stem cells, to enhance mobilization of hematopoietic stem cells, to improve the efficiency of targeting cells to the bone marrow and/or to modulate hematopoietic cell function.

### GOVERNMENT SUPPORT

The work leading to the present invention was funded in part by contract/grant numbers HL44851 and DK 50234 from the United States National Institutes of Health. Accordingly, the United States Government may have certain rights to this invention.

### FIELD OF THE INVENTION

The invention includes methods for manipulating hermatopoeitic stem cells and related products. In particular the invention includes methods and products for using stem cell G-protein coupled receptor (SC-GPR) related compositions to identify bone marrow derived hematopoietic stem cells, to enhance mobilization of hematopoietic stem cells, to improve the efficiency of targeting cells to the bone marrow and/or to modulate hematopoietic cell function.

### BACKGROUND OF THE INVENTION

Circulating blood cells, such as erythrocytes, leukocytes, platelets and lymphocytes, arise from the terminal differentiation of precursor cells, in a process referred to as hematopoiesis. In fetal life, hematopoiesis occurs throughout the reticular endothelial system. In the normal adult, terminal differentiation of the precursor cells occurs exclusively in the marrow cavities of the axial skeleton, with some extension into the proximal femora and humeri. These precursor cells, in turn, derive from immature cells, called progenitors, stem cells or hematopoietic cells.

Hematopoietic stem cells have therapeutic potential as a result of their capacity to restore blood and immune cell function in transplant recipients as well as their potential ability to generate cells for other tissues such as brain, muscle and liver (Choi, 1998 Biochem Cell Biol 76, 947-56; Eglitis and Mezey, 1997 Proc Natl Acad Sci USA 94, 4080-5; Gussoni et al., 1999 Nature 401, 390-4; Theise et al., 2000 Hepatology 32, 11-6). Human autologous and allogeneic bone marrow transplantation methods are currently used as therapies for diseases such as leukemia, lymphoma, and other life-threatening diseases. For these procedures a large amount of donor bone marrow must be isolated to ensure that there are enough cells for engraftment. Hematopoietic stem cell expansion for bone marrow transplantation is a potential method for generating human long-term bone marrow cultures for these therapeutic utilities. Several studies have reported the isolation and purification of hematopoietic stem cells (see, e.g., U.S. Pat. No. 5,061,620), but none of these methods have been overwhelmingly successful.

Determining the basis for stem cell localization is important to maximizing the therapeutic potential of those cells. For instance, the ability to manipulate stem cells could improve the efficiency of engraftment of transplanted cells. Currently, transplantation techniques are extremely inefficient. In view of their enormous therapeutic potential relatively little is known about how hematopoietic stem cells are regulated, e.g., what factors cause cell localization etc. Some studies have suggested that stem cell localization into the bone marrow space is chemokine dependent. For instance, the absence of either SDF-1 or its receptor, CXCR-4, was found to preclude localization of hematopoiesis in the bone marrow in developing mice (Nagasawa et al., 1996 Nature 382, 635-8; Su et al., 1999 J Immunol 162, 7128-7132; Zou et al., 1998 Nature 393, 595-9). In addition, manipulation of CXCR-4 alters the homing and retention of progenitors in adult mice further supporting its critical role (Ma et al., 1999. Immunity 10, 463-71; Peled et al., 1999 Science 283, 845-8). Selectins and integrins are also believed to participate in this process and have been identified as mediators of retention or adhesion of primitive cells to bone marrow in vivo or in vitro (Greenberg et al., 2000 Blood 95, 478-86; Naiyer et al., 1999 Blood 94, 4011-9; Rood et al., 1999 Exp Hematol 27, 1306-14; van der Loo et al., 1998 J Clin Invest 102, 1051-61; Williams et al., 1991 Nature 352, 438-41; Zanjani et al., 1999 Blood 94, 2515-22). These studies, however, have not provided a complete understanding of stem cell localization.

The ability of stem cells to survive through decades of life in contrast to short-lived progenitor populations has been attributed in part to their cytokine resistance and relative quiescence. While cycling does occur in the stem cell pool, the interval between doublings is dramatically different from the vigorously proliferative progenitor population. T1/2 for stem cell cycling has been estimated by BrdU labeling as 30 days in the mouse (Bradford et al., 1997 Exp Hematol 25, 445-53.) and, by population kinetics, as 10 weeks in the cat (Abkowitz et al., 1996 Nat Med 2, 190-7.). Recently the inhibition of cell cycle entry in stem cells has been proposed to be mediated by TGF-β signaling, but not MIP-lα or MCP-1 (Cashman et al., 1999 Blood 94, 3722-3729). We have recently recognized that stem cell cycling is restricted by the cyclin dependent kinase inhibitor, p21, and that the absence of p21 leads to both increased cycling and exhaustion of stem cell pools under conditions of stress (Cheng et al., 2000 Science 287, 1804-8.). Understanding exogenous signaling molecules which may contribute to the inhibition of entry into proliferative or differentiative pathways is important to defining targets for manipulation, and ultimately therapeutic procedures. Yet these studies are extremely difficult to perform due to the rarity of the cells and limited techniques for analysis.

### SUMMARY OF THE INVENTION

The invention relates in some aspects to methods for manipulating hematopoietic stem cells. Hematopoietic stem cells undergo a development stage-specific translocation during ontogeny and ultimately reside in the adult bone marrow. Maintenance of this highly regenerative cell pool through adult life is dependent upon their relative quiescence. It has been discovered according to the invention that a molecule referred to as stem cell G-protein coupled receptor or SC-GPR is involved in the regulation of hematopoietic stem cell properties such as quiescence and localization. This molecule is a seven transmembrane polypeptide, with a signature motif similar to a motif of the chemokine receptor family. It was discovered that antibodies raised against the SC-GPR gene product are useful for identifying cells from human fetal bone marrow and very rare cells from adult bone marrow (a subset of CD34⁺CD38⁻ cells). Additionally, these cells were found to be quiescent cell-enriched, with the ability to sustain mature blood cell generation for prolonged periods in either methylcellulose or on stromal feeder layers.

Thus, in one aspect the invention relates to a method for identifying a hematopoietic stem cell by identifying the presence or absence of a SC-GPR on a putative hematopoietic stem cell, the presence of the SC-GPR being indicative of a hematopoietic stem cell. In one embodiment the presence of SC-GPR is determined by contacting the putative hematopoietic stem cell with a SC-GPR binding agent. Optionally the SC-GPR binding agent is a SC-GPR binding peptide, such as, an anti- SC-GPR antibody or an anti-SC-GPR antibody fragment. In other embodiments the presence of SC-GPR is determined by contacting the putative hematopoietic stem cell with a SC-GPR nucleic acid probe. The putative hematopoietic stem cell may be present in a biological sample, such as a bone marrow sample or a blood sample.

In another aspect the invention is a method for isolating a hematopoietic stem cell by contacting a sample containing a hematopoietic stem cell with a SC-GPR binding agent to isolate the hematopoietic stem cell from the sample. In one embodiment the SC-GPR binding agent is not N-acetylglucosamine. In one embodiment the SC-GPR binding agent is a SC-GPR binding peptide, such as an anti-SC-GPR antibody or an anti-SC-GPR antibody fragment. The putative hematopoietic stem cell may be present in a biological sample, such as a bone marrow sample or a blood sample.

In one embodiment the hematopoietic stem cell is isolated using chromatography. Optionally the chromatography is column chromatography and the SC-GPR binding agent is fixed in the column. In other embodiments the hematopoietic stem cell is isolated using fluorescence activated cell sorting (FACS). In further embodiments the hematopoietic stem cell is isolated using magnetic bead technology.

In another aspect the invention is a kit including a SC-GPR binding agent, and instructions for contacting a hematopoietic stem cell with the SC-GPR binding agent to identify or isolate the hematopoietic stem cell. In one embodiment the SC-GPR binding agent is a SC-GPR binding peptide, such as an anti-SC-GPR antibody or an anti-SC-GPR antibody fragment. In another embodiment the SC-GPR binding agent is fixed to a chromatography matrix.

Transduction of SC-GPR nucleic acids into hematopoietic cells was found to enhance transmigration of the cells toward bone marrow stroma in vitro, and toward bone marrow in vivo. Thus, SC-GPR is useful for improving the efficiency of targeting transplanted stem cells to the bone marrow. It was also discovered that SC-GPR nucleic acid-transduced primary human progenitor cells acquire altered functional activity in vitro mimicking that of true stem cells, showing that expression of this receptor may confer stem cell characteristics to more mature progeny (e.g., “de-differentiation”).

Thus, in another aspect the invention relates to a SC-GPR-transduced hematopoietic cell. In one embodiment the SC-GPR-transduced hematopoietic cell is prepared by transduction of a SC-GPR-negative hematopoietic cell with a nucleic acid of SEQ ID No. 10. In another embodiment the cell also includes an exogenous gene encoding a therapeutic agent.

According to another aspect of the invention a method for supplementing bone marrow is provided. The method involves administering to a subject a SC-GPR enriched hematopoietic cell population to supplement the bone marrow of the subject. In one embodiment the SC-GPR enriched hematopoietic cell population includes SC-GPR-transduced hematopoietic cells. In other embodiments the SC-GPR enriched hematopoietic cell population includes hematopoietic cells isolated by the method of the invention. The subject, in some embodiments is a subject in need of a bone marrow transplant. Optionally the SC-GPR enriched hematopoietic cell population is administered to the peripheral blood of the subject.

SC-GPR antagonists or blocking agents were found to be useful to enhance mobilization of hematopoietic stem cells. Such effect is highly desirable in current practices of bone marrow transplantation, where a donor's bone marrow cells could be “mobilized” and stem cells can be easily isolated from the donor's peripheral blood. Thus, according to another aspect the invention relates to a method for enhancing mobilization of hematopoietic stem cells, by administering to a subject a SC-GPR inhibitor to enhance mobilization of hematopoietic stem cells in the subject. In some embodiments the SC-GPR inhibitor is a SC-GPR antagonist, a SC-GPR blocking agent, or a SC-GPR antisense molecule. In another embodiment the subject is a bone marrow donor.

A method for modulating hematopoietic cell function, is provided according to another aspect of the invention. The method involves contacting a hematopoietic cell with a SC-GPR activator or a SC-GPR inhibitor to modulate hematopoietic cell function. In some embodiments the SC-GPR inhibitor is a SC-GPR antagonist, a SC-GPR blocking agent, or a SC-GPR antisense molecule. In other embodiments the SC-GPR activator is a SC-GPR nucleic acid or a SC-GPR agonist.

Surprisingly, according to the invention, it has been discovered that under appropriate conditions, SC-GPR-transduced hematopoietic cells are capable of forming an array of cell and tissue types, including mesenchymal, parenchymal, neuronal, endothelial, and epithelial cells.

The invention has therefore a variety of therapeutic applications in tissue repair, tissue transplantation, tissue re-implantation, tissue-specific expression of recombinant genes, and the like. Examples of such tissues include, but are not limited to, brain tissue, breast tissue, gastrointestinal tissue, ovarian tissue, and/or tissue of the following organs and/or systems: Blood and Blood Forming system: including platelets, blood vessel wall, and bone marrow; Cardiovascular system: including heart and vascular system; Digestive and excretory system: including alimentary tract, biliary tract, kidney, liver, pancreas and urinary tract; Endocrine system: including adrenal gland, kidney, ovary, pituitary gland, renal gland, salivary gland, sebaceous gland, testis, thymus gland and thyroid gland; Muscular system: including muscles that move the body; Reproductive System: including breast, ovary, penis and uterus; Respiratory system: including bronchus, lung and trachea; Skeletal system: including bones and joints; Tissue, fiber, and integumentary system: including adipose tissue, cartilage, connective tissue, cuticle, dermis, epidermis, epithelium, fascia, hair follicle, ligament, bone marrow, melanin, melanocyte, mucous membrane, skin, soft tissue, synovial capsule and tendon.

According to one aspect of the invention, a method for in vitro culture of hematopoietic cells to produce differentiated cells of non-hematopoietic lineage, is provided. The method involves contacting a hematopoietic cell with a SC-GPR activator (e.g., by transduction to produce a SC-GPR-transduced hematopoietic cell) under conditions sufficient to confer hematopoietic stem cell characteristics (or properties) to the hematopoietic cell, and culturing the hematopoietic cell having stem cell characteristics in an environment that promotes hematopoietic cell differentiation, under conditions and for a period of time to produce differentiated cells of non-hematopoietic lineage. In some embodiments the SC-GPR activator is a SC-GPR nucleic acid. In certain embodiments the SC-GPR activator is a SC-GPR agonist. The contacting may occur in vivo or in vitro.

According to still another aspect of the invention, a method for inducing hematopoietic stem cell quiescence is provided. The method involves contacting a hematopoietic stem cell with an effective amount of a SC-GPR activator to induce quiescence of the hematopoietic stem cell. In some embodiments the SC-GPR activator is a SC-GPR nucleic acid. In certain embodiments the SC-GPR activator is a SC-GPR agonist. The contacting may occur in vivo or in vitro.

According to another aspect of the invention, a method for inhibiting hematopoietic stem cell-death is provided. The method involves inducing hematopoietic stem cell quiescence according to any of the methods described in the preceding paragraph to inhibit hematopoietic stem cell-death. In important embodiments the hematopoietic stem cell is under environmental stress. Environmental stresses include increased temperatures (e.g., fever), physical trauma, oxidative, osmotic and chemical stress (e.g. a chemotherapeutic agent), and UV irradiation.

Each of the limitations of the invention can encompass various embodiments of the invention. It is, therefore, anticipated that each of the limitations of the invention involving any one element or combinations of elements can be included in each aspect of the invention.

### BRIEF DESCRIPTION OF THE SEQUENCES

SEQ ID NO.: 1: human SC-GPR amino acid sequence.

SEQ ID NO.: 2: mouse SC-GPR amino acid sequence.

SEQ ID NO.: 3: rat SC-GPR amino acid sequence.

SEQ ID NO.: 4: a peptide (named N-SC-GPR, MINSTSTQPPDESCSQN).

SEQ ID NO.: 5: DRYYKIV.

SEQ ID NO.: 6: DRYLAIV.

SEQ ID NO.: 7: primer SC-GPR-sBam 5′-CGG GAT CCC GAA GTT ACA AGA TGA TCA ATT CAA CC.

SEQ ID NO.: 8: primer SC-GPR-aXho 5′-CCG CTC GAG CGG AAG AGG GTA GGA ACT CA.

SEQ ID NO.: 9: YPYDVPDYA.

SEQ ID NO.: 10: human SC-GPR nucleic acid sequence.

### DETAILED DESCRIPTION OF THE INVENTION

New methods for manipulating hematopoietic stem cells have been identified according to the invention. These methods and related products have great therapeutic and research value. For instance, hematopoietic stem cells are used for transplantation to supplement the immune system of immune deficient patients. These cells have many additional therapeutic uses. Prior to the invention, however, the ability to isolate and purify hematopoietic stem cells has been limited. These cells reside in the bone marrow, making their isolation a technically complex procedure. Additionally, there are not many commercially viable methods for identifying these cells in a sample. The invention has solved many of these problems.

It has been discovered according to the invention that SC-GPR is preferentially expressed in quiescent bone marrow derived primitive hematopoietic cells and has characteristics suggesting it is a member of the chemokine receptor family; a group of molecules known to mediate both localization and anti-proliferative phenomena in hematopoietic cell systems. Antiserum raised against this receptor may be used to isolate a population of cells from primary tissue with the ability to sustain blood cell production over weeks to months in vitro. The receptor is responsive to bone marrow microenviromnental cues, demonstrating intracellular calcium flux and transmigration to bone marrow stroma or stroma conditioned medium. When ectopically expressed the receptor mediates bone marrow homing in vivo and alters differentiation kinetics of primary hematopoietic cells.

Current practice relating to hematopoietic stem cell isolation involves a small number of cell surface antigens (e.g., CD34, CD38, etc.), which can give inconsistent results. Antibodies to SC-GPR or other SC-GPR binding agents identify rare adult bone marrow cells and more abundant fetal bone marrow cells, which are highly enriched for hematopoietic stem cells (as assessed by functional assays) and are quiescent (a property of long term repopulating stem cells). Therefore, SC-GPR binding agents represent a novel means for isolating a stem cell pool that is enriched beyond that possible with existing technologies.

Thus, in some aspects the invention is a method for identifying a hematopoietic stem cell. The method involves identifying the presence or absence of a SC-GPR on a putative hematopoietic stem cell, the presence of the SC-GPR being indicative of a hematopoietic stem cell.

The cells used according to the methods of the invention are hematopoietic stem cells. “Hematopoietic stem cells” as used herein refers to immature blood cells having the capacity to self-renew and to differentiate into mature blood cells comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), and monocytes (e.g., monocytes, macrophages). Cells of “hematopoietic origin” include, but are not limited to, pluripotent stem cells, multipotent progenitor cells and/or progenitor cells committed to specific hematopoietic lineages. The progenitor cells committed to specific hematopoietic lineages may be of T cell lineage, B cell lineage, dendritic cell lineage, Langerhans cell lineage and/or lymphoid tissue-specific macrophage cell lineage.

The hematopoietic stem cells can be obtained from biological samples such as blood products. A “blood product” as used herein is a product obtained from the body or an organ of the body containing cells of hematopoietic origin. Such sources include unfractionated bone marrow, umbilical cord, skin, brain, peripheral blood, liver, thymus, lymph and spleen. In some embodiments the biological sample is bone marrow or peripheral blood. Unfractionated blood products can be obtained directly from a donor or retrieved from cryopreservative storage.

The hematopoietic stem cells can be identified by determining the presence or absence of SC-GPR on a putative cell. “SC-GPR” as used herein refers to a seven transmembrane polypeptide (or nucleic acid encoding the same) also known as stem cell G-protein coupled receptor. SC-GPR has a signature motif similar to a motif of the chemokine receptor family and with a nucleic acid sequence identical to the sequence of a previously identified gene, KIAA0001 (GenBank accession number D13626 or NM₁₃ 014879, SEQ ID No.:10). KIAA0001 was originally isolated from a cDNA library of human immature myeloid cell line KG-1 (Numura, N. et al. DNA Research, 1994, 1:47-56) and was characterized as a G-protein coupled receptor. It was not until recently that a function was assigned to this molecule (Chambers, J K et al., J. Biol. Chem., 2000, 275:1067-71). According to this report, KIAA0001 was identified as a G-protein coupled receptor for UDP-glucose. The human nucleic acid and human, mouse and rat peptide sequences of native SC-GPR are set forth in SEQ ID NO 10, 1, 2, and 3 respectively.

The method involves detecting the presence of a SC-GPR in a cell. The presence of SC-GPR in a cell indicates that the cell is a hematopoietic cell. Optionally, the presence of SC-GPR may be measured in the cell by contacting the cell with a SC-GPR binding agent that selectively binds to the SC-GPR to detect or measure the presence of the SC-GPR in the cell. SC-GPR can be detected by standard methods of gene and protein detection. For instance, methods for detection and/or quantitation of gene expression include methods of detecting specific mRNA either quantitatively with Northern blots, S1 nuclease, RNAse protection, RT-PCR, or localization detection means such as in situ hybridization and in situ PCR Methods for quantitation of peptide levels in a given tissue may be measured using radioimmunoassay (RIA), enzyme-linked immunosorbent assays (ELISA), and immuno PCR or localized by immunohistochemistry.

When the SC-GPR is a SC-GPR mRNA, the detection reagent can be a SC-GPR nucleic acid probe that selectively hybridizes to the SC-GPR mRNA. According to this embodiment, the cell is contacted with the detection reagent under conditions that permit selective hybridization of the SC-GPR nucleic acid probe to the SC-GPR mRNA. A “SC-GPR nucleic acid probe”, as used herein, refers to a nucleic acid molecule which hybridizes under stringent conditions to a nucleic acid having the sequence of SEQ ID NO: 10 or variants or homologs or unique fragments thereof. Such unique fragments can be used, for example, as probes in hybridization assays and as primers in a polymerase chain reaction (PCR) in order to detect the presence of SC-GPR mRNA. A unique fragment is one that is a ‘signature’ for the larger nucleic acid. It, for example, is long enough to assure that its precise sequence is not found in molecules outside of the SC-GPR gene. A preferred SC-GPR nucleic acid probe for this embodiment is a SC-GPR nucleic acid probe having a sequence complementary to SEQ ID NO:10 or a unique fragment thereof.

Alternatively, the SC-GPR that is being assayed can be a SC-GPR polypeptide and the SC-GPR binding agent selectively binds to the SC-GPR polypeptide. In some embodiments the SC-GPR binding agent may be a SC-GPR binding peptide such as an anti-SC-GPR antibody or fragment that selectively binds to the SC-GPR polypeptide. The SC-GPR polypeptide can be contacted with the SC-GPR binding agent under conditions that permit selective binding of the binding agent to the SC-GPR polypeptide. The SC-GPR may optionally be separated from the intact cell in the form of isolated nucleic acid or polypeptide fractions prior to the detection step.

The invention also includes methods for isolating a hematopoietic stem cell, by contacting a sample containing a hematopoietic stem cell with a SC-GPR binding agent to isolate the hematopoietic stem cell from the sample.

A “SC-GPR binding agent”, as used herein, refers to a molecule which interacts with SC-GPR, and includes but is not limited to antibodies, antibody fragments, other peptides, mimetics, etc. An “SC-GPR binding peptide” as used herein refers to a peptide or a fragment thereof that selectively binds to an epitope of the SC-GPR The SC-GPR binding agents of the invention can be identified using routine assays, such as the binding and activation assays described throughout this patent application.

The SC-GPR binding agent is an isolated molecule. An isolated molecule is a molecule that is substantially pure and is free of other substances with which it is ordinarily found in nature or in vivo systems to an extent practical and appropriate for its intended use. In particular, the molecular species are sufficiently pure and are sufficiently free from other biological constituents of host cells so as to be useful in, for example, producing pharmaceutical preparations or sequencing if the molecular species is a nucleic acid, peptide, or polysaccharide. Because an isolated molecular species of the invention may be admixed with a pharmaceutically-acceptable carrier in a pharmaceutical preparation, the molecular species may comprise only a small percentage by weight of the preparation. The molecular species is nonetheless substantially pure in that it has been substantially separated from the substances with which it may be associated in living systems.

The SC-GPR binding agents may be isolated from natural sources or synthesized or produced by recombinant means. Methods for preparing or identifying agents which bind to a particular target are well-known in the art. Molecular imprinting, for instance, may be used for the de novo construction of macro molecular structures, such as peptides, which bind to a particular molecule. See for example, Kenneth J. Shea, Molecular Imprinting of Synthetic Network Polymers: The De Novo Synthesis of Molecular Binding In Catalytic Sites, Trip, to May 1994; Klaus, Mosbach, Molecular Imprinting, Trends in Biochem. Sci., 19(9), January 1994; and Wulff, G., In Polymeric Reagents and Catalysts (Ford, W. T., ed.) ACS Symposium Series No. 308, P.186-230, Am. Chem. Soc. 1986. Binding peptides, such as antibodies, may easily be prepared by generating antibodies to SC-GPR (or obtained from commercial sources) or by screening libraries to identify peptides or other compounds which bind to the SC-GPR.

Mimics of known binding agents may also be prepared by known methods, such as (i) polymerization of functional monomers around a known binding agent or the binding region of an antibody which also binds to the target (the template) that exhibits the desired activity; (ii) removal of the template agent; and then (iii) polymerization of a second class of monomers in the void left by the template, to provide a new agent which exhibits one or more desired properties which are similar to that of the template. The method is useful for preparing peptides, and other binding agents which have the same function as binding peptides, such as polysaccharides, nucleotides, nucleoproteins, lipoproteins, carbohydrates, glycoproteins, steroids, lipids and other biologically-active material can also be prepared. Thus a template, such as a known SC-GPR binding peptide can be used to identify SC-GPR binding agents. It is now routine to produce large numbers of binding agents based on one or a few peptide sequences or sequence motifs. (See, e.g., Bromme, et al., Biochem. J. 315:85-89 (1996); Palmer, et al., J. Med Chem. 38:3193-3196 (1995)). For example, if SC-GPR is known to interact with protein X at position Y, a binding agent of SC-GPR may be chosen or designed as a polypeptide or modified polypeptide having the same sequence as protein X, or structural similarity to the sequence of protein X, in the region adjacent to position Y. In fact, the region adjacent to the cleavage site Y spanning residues removed by 10 residues or, more preferably 5 residues, N-terminal and C-terminal of position Y, may be defined as a “preferred protein X site” for the choice or design of SC-GPR binding agents. Thus, a plurality of SC-GPR binding agents chosen or designed to span the preferred protein X binding site around position Y, may be produced, tested for activity, and sequentially modified to optimize or alter activity, stability, and/or specificity.

The method is useful for designing a wide variety of biological mimics that are more stable than the natural counterpart, because they are typically prepared by the free radical polymerization of functional monomers, resulting in a compound with a non-biodegradable backbone. Thus, the created molecules may have the same binding properties as the known SC-GPR peptide but be more stable in vivo, thus preventing SC-GPR from interacting with components normally available in its native environment. Other methods for designing such molecules include, for example, drug design based on structure activity relationships which require the synthesis and evaluation of a number of compounds and molecular modeling.

Binding agents may also be identified by conventional screening methods, such as phage display procedures (e.g. methods described in Hart et al., J. Biol Chem. 269:12468 (1994)). Hart et al. report a filamentous phage display library for identifying novel peptide ligands. In general, phage display libraries using, e.g., M13 or fd phage, are prepared using conventional procedures such as those described in the foregoing reference. The libraries generally display inserts containing from 4 to 80 amino acid residues. The inserts optionally represent a completely degenerate or biased array of peptides. Ligands having the appropriate binding properties are obtained by selecting those phage which express on their surface a ligand that binds to the target molecule. These phage are then subjected to several cycles of reselection to identify the peptide ligand expressing phage that have the most useful binding characteristics. Typically, phage that exhibit the best binding characteristics (e.g., highest affinity) are further characterized by nucleic acid analysis to identify the particular amino acid sequences of the peptide expressed on the phage surface in the optimum length of the express peptide to achieve optimum binding.

Alternatively, SC-GPR binding agents can be identified from combinatorial libraries. Many types of combinatorial libraries have been described. For instance, U.S. Pat. No. 5,712,171 (which describes methods for constructing arrays of synthetic molecular constructs by forming a plurality of molecular constructs having the scaffold backbone of the chemical molecule and modifying at least one location on the molecule in a logically-ordered array); U.S. Pat. No. 5,962,412 (which describes methods for making polymers having specific physiochemical properties); and U.S. Pat. No. 5,962,736 (which describes specific arrayed compounds).

To determine whether an agent binds to the appropriate target any known binding assay may be employed. For example, in the case of a peptide that binds to the plasma membrane SC-GPR the agent may be immobilized on a surface and then contacted with a labeled plasma membrane SC-GPR (or vice versa). The amount of plasma membrane SC-GPR which interacts with the agent or the amount which does not bind to the agent may then be quantitated to determine whether the agent binds to plasma membrane SC-GPR. A surface having a known agent that binds to plasma membrane SC-GPR such as a monoclonal antibody immobilized thereto may serve as a positive control.

Screening of agents of the invention, also can be carried out utilizing a competition assay. If the agent being tested competes with the known monoclonal antibody, as shown by a decrease in binding of the known monoclonal antibody, then it is likely that the agent and the known monoclonal antibody bind to the same, or a closely related, epitope. Still another way to determine whether a molecule has the specificity of the known monoclonal antibody is to pre-incubate the known monoclonal antibody with the target with which it is normally reactive, and then add the agent being tested to determine if the agent being tested is inhibited in its ability to bind the target. If the agent being tested is inhibited then, in all likelihood, it has the same, or a functionally equivalent, epitope and specificity as the known monoclonal antibody.

By using a SC-GPR monoclonal antibody, it is also possible to produce anti-idiotypic antibodies which can be used to screen other antibodies to identify whether the antibody has the same binding specificity as the known monoclonal antibody. Such anti-idiotypic antibodies can be produced using well-known hybridoma techniques (Kohler and Milstein, Nature, 256:495, 1975). An anti-idiotypic antibody is an antibody which recognizes unique determinants present on the known monoclonal antibodies. These determinants are located in the hypervariable region of the antibody. It is this region which binds to a given epitope and, thus, is responsible for the specificity of the antibody. An anti-idiotypic antibody can be prepared by immunizing an animal with the known monoclonal antibodies. The immunized animal will recognize and respond to the idiotypic determinants of the immunizing known monoclonal antibodies and produce an antibody to these idiotypic determinants. By using the anti-idiotypic antibodies of the immunized animal, which are specific for the known monoclonal antibodies of the invention, it is possible to identify other clones with the same idiotype as the known monoclonal antibody used for immunization. Idiotypic identity between monoclonal antibodies of two cell lines demonstrates that the two monoclonal antibodies are the same with respect to their recognition of the same epitopic determinant. Thus, by using anti-idiotypic antibodies, it is possible to identify other hybridomas expressing monoclonal antibodies having the same epitopic specificity.

It is also possible to use the anti-idiotype technology to produce monoclonal antibodies which mimic an epitope. For example, an anti-idiotypic monoclonal antibody made to a first monoclonal antibody will have a binding domain in the hypervariable region which is the image of the epitope bound by the first monoclonal antibody.

In one embodiment the binding peptides useful according to the invention are antibodies or functionally active antibody fragments. Antibodies are well known to those of ordinary skill in the science of immunology. The binding peptides described herein may be used as intact functional antibodies. As used herein, the term “antibody” means not only intact antibody molecules but also fragments of antibody molecules retaining specific binding ability. Such fragments are also well known in the art and are regularly employed both in vitro and in vivo. In particular, as used herein, the term “antibody” means not only intact immunoglobulin molecules but also the well-known active fragments F(ab′)₂, and Fab. F(ab′)₂, and Fab fragments which lack the Fc fragment of intact antibody, clear more rapidly from the circulation, and may have less non-specific tissue binding of an intact antibody (Wahl et al., J. Nucl. Med. 24:316-325 (1983)).

As is well-known in the art, the complementarity determining regions (CDRs) of an antibody are the portions of the antibody which are largely responsible for antibody specificity. The CDR's directly interact with the epitope of the antigen (see, in general, Clark; 1986; Roitt, 1991). In both the heavy chain and the light chain variable regions of IgG immunoglobulins, there are four framework regions (FR1 through FR4) separated respectively by three complementarity determining regions (CDR1 through CDR3). The framework regions FRs) maintain the tertiary structure of the paratope, which is the portion of the antibody which is involved in the interaction with the antigen. The CDRs, and in particular the CDR3 regions, and more particularly the heavy chain CDR3 contribute to antibody specificity. Because these CDR regions and in particular the CDR3 region confer antigen specificity on the antibody these regions may be incorporated into other antibodies or peptides to confer the identical specificity onto that antibody or peptide.

According to one embodiment, the peptide of the invention is an intact soluble monoclonal antibody in an isolated form or in a pharmaceutical preparation. An intact soluble monoclonal antibody, as is well known in the art, is an assembly of polypeptide chains linked by disulfide bridges. Two principle polypeptide chains, referred to as the light chain and heavy chain, make up all major structural classes (isotypes) of antibody. Both heavy chains and light chains are further divided into subregions referred to as variable regions and constant regions. As used herein the term “monoclonal antibody” refers to a homogenous population of immunoglobulins which specifically bind to an epitope (i.e. antigenic determinant), e.g., of SC-GPR.

The peptide useful according to the methods of the present invention may be an intact humanized a monoclonal antibody. A “humanized monoclonal antibody” as used herein is a human monoclonal antibody or functionally active fragment thereof having human constant regions and a binding CDR3 region from a mammal of a species other than a human. Humanized monoclonal antibodies may be made by any method known in the art. Humanized monoclonal antibodies, for example, may be constructed by replacing the non-CDR regions of a non-human mammalian antibody with similar regions of human antibodies while retaining the epitopic specificity of the original antibody. For example, non-human CDRs and optionally some of the framework regions may be covalently joined to human FR and/or Fc/pFc′ regions to produce a functional antibody. There are entities in the United States which will synthesize humanized antibodies from specific murine antibody regions commercially, such as Protein Design Labs (Mountain View Calif.).

European Patent Application 0239400, the entire contents of which is hereby incorporated by reference, provides an exemplary teaching of the production and use of humanized monoclonal antibodies in which at least the CDR portion of a murine (or other non-human mammal) antibody is included in the humanized antibody. Briefly, the following methods are useful for constructing a humanized CDR monoclonal antibody including at least a portion of a mouse CDR. A first replicable expression vector including a suitable promoter operably linked to a DNA sequence encoding at least a variable domain of an Ig heavy or light chain and the variable domain comprising framework regions from a human antibody and a CDR region of a murine antibody is prepared. Optionally a second replicable expression vector is prepared which includes a suitable promoter operably linked to a DNA sequence encoding at least the variable domain of a complementary human Ig light or heavy chain respectively. A cell line is then transformed with the vectors. Preferably the cell line is an immortalized mammalian cell line of lymphoid origin, such as a myeloma, hybridoma, trioma, or quadroma cell line, or is a normal lymphoid cell which has been immortalized by transformation with a virus. The transformed cell line is then cultured under conditions known to those of skill in the art to produce the humanized antibody.

As set forth in European Patent Application 0239400 several techniques are well known in the art for creating the particular antibody domains to be inserted into the replicable vector. (Preferred vectors and recombinant techniques are discussed in greater detail below.) For example, the DNA sequence encoding the domain may be prepared by oligonucleotide synthesis. Alternatively a synthetic gene lacking the CDR regions in which four framework regions are fused together with suitable restriction sites at the junctions, such that double stranded synthetic or restricted subcloned CDR cassettes with sticky ends could be ligated at the junctions of the framework regions. Another method involves the preparation of the DNA sequence encoding the variable CDR containing domain by oligonucleotide site-directed mutagenesis. Each of these methods is well known in the art. Therefore, those skilled in the art may construct humanized antibodies containing a murine CDR region without destroying the specificity of the antibody for its epitope.

Human monoclonal antibodies may be made by any of the methods known in the art, such as those disclosed in U.S. Pat. No. 5,567,610, issued to Borrebaeck et al., U.S. Pat. No. 565,354, issued to Ostberg, U.S. Pat. No. 5,571,893, issued to Baker et al, Kozber, J. Immunol. 133: 3001 (1984), Brodeur, et al., Monoclonal Antibody Production Techniques and Applications, p. 51-63 (Marcel Dekker, Inc, new York, 1987), and Boerner el al., J. Immunol., 147: 86-95 (1991). In addition to the conventional methods for preparing human monoclonal antibodies, such antibodies may also be prepared by immunizing transgenic animals that are capable of producing human antibodies (e.g., Jakobovits et al., PNAS USA, 90: 2551 (1993), Jakobovits et al., Nature, 362: 255-258 (1993), Bruggermann et al., Year in Immuno., 7:33 (1993) and U.S. Pat. No. 5,569,825 issued to Lonberg).

The binding peptides may also be functionally active antibody fragments. Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. An isolated F(ab′)₂ fragment is referred to as a bivalent monoclonal fragment because of its two antigen binding sites. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd (heavy chain variable region). The Fd fragments are the major determinant of antibody specificity (a single Fd fragment may be associated with up to ten different light chains without altering antibody specificity) and Fd fragments retain epitope-binding ability in isolation.

The terms Fab, Fc, pFc′, F(ab′)₂ and Fv are used consistently with their standard immunological meanings [Klein, Immunology (John Wiley, New York, N.Y., 1982); Clark, W. R. (1986) The Experimental Foundations of Modem Immunology (Wiley & Sons, Inc., New York); Roitt, I. (1991) Essential Immunology, 7th Ed., (Blackwell Scientific Publications, Oxford)].

One method for accomplishing the isolation of a hematopoietic stem cell is through the use of fluorescence-activated cell sorting (FACS, e.g., using a flow cytometer, i.e., FACScan, Becton Dickinson, San Jose, Calif.). Flow cytometry sorts cells one at a time and physically separates one set of labeled cells from another second set of cells. This widely described method makes use of sophisticated equipment comprising a liquid flux in which the cells move. FACS analysis involves the separation of cells based on the identification of a fluorescent tag on the cell surface. Thus, a flourescently labeled molecule such as a binding peptide which emits light in a particular wavelength will be recognized and separated from other components. If the labeled binding peptide is attached to a cell then the cell will be separated. A laser beam stimulates the fluorescence and thus sets off a signal which makes it possible to divert the cell electrically into a container. This method if very effective and makes it possible to achieve an enrichment of nearly 100%.

Milner et al., 1994, Blood 83:2057-2062 describes a method for isolating hematopoietic stem cells from bone marrow. Bone marrow samples are obtained and separated by Ficoll-Hypaque density gradient centrifugation. They are then washed and stained using two-color indirect immunofluorescent antibody binding and separated by FACS. The cells are labeled simultaneously with IgG antibodies such-that CD34⁺ hematopoietic stem cells, including the immature subset that lacks expression of individual lineage associated antigens, CD34^{+/-}, are isolated from the cells collected from marrow. The methods of the invention may be performed in a similar manner using a labeled SC-GPR binding agent rather than CD34 binders. Also, Gazitt et al. used FACS to sort hematopoietic stem cells from tumor cells (Gazitt et al. Blood, 86(1):381-389, 1995).

One method for accomplishing the isolation of a hematopoietic stem cell is through the use of chromatography such as affinity or immunoaffinity chromatography. Other devices for separating cells on the basis of the presence of a cell surface protein have been described. See for example, U.S. Pat. Nos. 6,069,014 and 6,013,531.

Optionally, the isolated hematopoietic stem cell can be grown in culture. It is possible to preserve the isolated hematopoietic stem cells and to stimulate the expansion of hematopoietic stem cells in vitro after isolation. Once expanded, the cells, for example, can be returned to the body to supplement, replenish, etc. a patient's hematopoietic stem cell population. This might be appropriate, for example, after an individual has undergone chemotherapy. There are certain genetic conditions wherein hematopoietic stem cell numbers are decreased, and the methods may be used in these situations as well.

As shown in the Examples, SC-GPR+ bone marrow cells provide ongoing hematopoietic cell output in co-culture. The cells sustain production of mature blood cells over months of co-culture and do so with substantially less attrition than that seen in preparations of CD34+ CD38− cells. The long-term culture systems were developed specifically to deplete the contribution of more mature populations by the time the end points of the assay were achieved. The limited early production of cells by the SC-GPR+ population, yet persistent production over long intervals suggests that these cells can be manipulated for long periods of time in culture prior to transplantation.

It also is possible to further manipulate the hematopoietic stem cells isolated according to the invention e.g., with hematopoietic growth agents that promote hematopoietic cell differentiation, to yield the more mature blood cells, in vitro. Such expanded populations of blood cells may be applied in vivo to a subject, or may be used experimentally as will be recognized by those of ordinary skill in the art. Such differentiated cells include those described above.

Methods for maintaining and manipulating hematopoietic stem cells in vitro, prior to transplantation or for experimental purposes have been described extensively in the prior art. For example, PCT published patent application numbers WO 99/15629 and WO 00/27999 describe improved methods for growing and expanding hematopoietic stem cells. Long-term cultures of bone marrow cells can be established and maintained by using, for example, modified Dexter cell culture techniques (Dexter et al., 1977, J. Cell Physiol. 91:335) or Witlock-Witte culture techniques (Witlock and Witte, 1982, Proc. Natl. Acad. Sci. USA 79:3608-3612). Briefly, methods for culturing cells and the media used are those conventionally used in the art. Examples of conventional media include RPMI, DMEM, ISCOVES, etc. Typically these media are supplemented with human or animal plasma or serum. Such plasma or serum can contain small amounts of hematopoietic growth factors. Hematopoietic growth factors, are secreted factors that influence the survival, proliferation or differentiation of hematopoietic cells. Growth agents that affect only survival and proliferation, but are not believed to promote differentiation, include the interleukin (IL)3, IL6 and IL11, stem cell ligand and FLT ligand. Hematopoietic growth factors that promote differentiation include the colony stimulating factors such as GMCSF, GCSF, MCSF and interleukins other than IL3, IL6 and IL11. The foregoing factors are well known to those of ordinary skill in the art. Most are commercially available. They can be obtained by purification, by recombinant methodologies or can be derived or synthesized synthetically.

The hematopoietic stem cells may also be cultured in an environment that includes inoculated stromal cells or stromal cell conditioned medium. “Inoculated” stromal cells, promote survival, proliferation or differentiation of the hematopoietic stem cells. “Stromal cells” as used herein comprise fibroblasts and mesenchymal cells, with or without other cells and elements, and can be seeded prior to, or substantially at the same time as, the hematopoietic stem cells, therefore establishing conditions that favor the subsequent attachment and growth of hematopoietic stem cells. Fibroblasts can be obtained via a biopsy from any tissue or organ, and include fetal fibroblasts. These fibroblasts and mesenchymal cells may be transfected with exogenous DNA that encodes for example one of the hematopoietic growth factors described above.

“Stromal cell conditioned medium” refers to medium in which the aforementioned stromal cells have been incubated. The incubation is performed for a period sufficient to allow the stromal cells to secrete factors into the medium. Such “stromal cell conditioned medium” can then be used to supplement the culture of hematopoietic stem cells promoting their proliferation and differentiation.

In another aspect, the invention includes a kit for identifying or isolating a hematopoietic stem cell. The kit may be in one or more containers and, preferably, includes any of the above-noted reagents as well as instructions for carrying out the methods. Optionally, the kit further includes additional reagents useful in the identification and isolation methods, such as PCR, or blot reagents or chromatography matrix.

The invention also relates to methods for modulating hematopoietic cell function. The methods may be accomplished by contacting a hematopoietic cell with a SC-GPR activator or a SC-GPR inhibitor to modulate hematopoietic cell function. As used herein “modulating hematopoietic cell function” refers to causing any change in the mobilization, migration or differentiation properties of a hematopoietic stem cell. For example, it has been discovered, unexpectedly, that when a hematopoietic cell of a mature phenotype (i.e. not having stem cell characteristics) is activated with SC-GPR (e.g., transduced with a SC-GPR nucleic acid or polypeptide, the mature hematopoietic cell de-differentiates and acquires stem cell characteristics.

It has also been discovered, unexpectedly, that when a hematopoietic stem cell is contacted with an exogenous SC-GPR molecule (e.g. transduced), or SC-GPR (endogenous) is activated in the hematopoietic stem cell, the hematopoietic stem cell becomes quiescent. A “quiescent stem cell” refers to a stem cell in the G₁ or G₀ phase of the cell cycle. A population of cells is considered herein to be a population of quiescent cells when at least 50%, preferably at least 70%, more preferably at least 80% of the cells are in the G₁ or G₀ phase of the cell cycle. Quiescent cells exhibit a single DNA peak by flow-cytometry analysis, a standard technique well known to those of ordinary skill in the arts of immunology and cell biology. Another technique useful for determining whether a population of cells is quiescent is the addition of a chemical agent to the cell culture medium that is toxic only to actively cycling cells, i.e., DNA synthesizing cells, and does not kill quiescent cells. Non-exclusive examples of such chemical agents include hydroxyurea and high specific activity tritiated thymidine (³HtdR). A population of cells is evaluated as to the percent in an actively cycling state by the percent of the cell population killed by the chemical agent. A cell population in which in vitro tritiated thymidine killing is less than approximately 30%, preferably less than approximately 10%, more preferably less than approximately 5%, is considered to be quiescent.

According to another aspect of the invention, a method for inhibiting hematopoietic stem cell-death is provided, particularly when the hematopoietic stem cell is subjected to an environmental stress. The method involves inducing hematopoietic stem cell quiescence by contacting the cell with a SC-GPR activator prior to or during the application of the stress, both in vivo and in vitro. The lifespan of a hematopoietic stem cell (or any other mammalian lo cell) under environmental stress is significantly shorter when compared to the lifespan of a hematopoietic stem cell under no such stress. This can be easily detected by placing a number of cells under a form of environmental stress and comparing their survival (numbers) to an identical number of cells free from any stress over a period of time. The amount of the foregoing agent(s) of the invention sufficient to inhibit cell-death, is the amount sufficient to extend the lifespan of the hematopoietic stem cell under environmental stress toward comparable lifespan lengths of hematopoietic stem cells free from any environmental stress. Such methods can be used to protect cells from environmental insults, such as increased temperatures (e.g., fever), physical trauma, oxidative, osmotic and chemical stress(e.g. a chemotherapeutic agent), and UV irradiation.

In some embodiments the SC-GPR inhibitor is a SC-GPR antagonist, a SC-GPR blocking agent, or a SC-GPR antisense agent. In other embodiments the SC-GPR activator is a SC-GPR nucleic acid or a SC-GPR agonist. The SC-GPR inhibitors and activators may be SC-GPR binding agents, but do not necessarily have to be.

One method for modulating hematopoietic cell function is a method for enhancing mobilization of hematopoietic stem cells by using SC-GPR inhibitors. Current practice during bone marrow transplantation involves the isolation of bone marrow cells from the bone marrow and/or peripheral blood of donor subjects. About one third of these subjects do not “yield” enough hematopoietic progenitor cells from their bone marrow and/or peripheral blood so that their marrow can be considered suitable for transplantation. Using the methods of the invention, the “yield” may be enhanced. For example, SC-GPR inhibitors which block the function of the receptor (i.e., block targeting of SC-GPR-expressing hematopoietic stem cells to the bone marrow) could result in “mobilization” of hematopoietic stem cells and the efficiency of hematopoietic progenitor cell isolation from subjects treated with such inhibitors may be improved (especially from the subject's peripheral blood). This then results in an increase in the number of donor samples that may be used in transplantation.

The importance of SC-GPR in mediating marrow specific localization was initially suggested by its expression within a subset of stem cells derived from fetal bone marrow during a time in human ontogeny of active stem cell translocation. Direct evidence of movement of cells bearing the receptor to bone marrow stroma in vitro and homing of such cells to bone marrow in vivo confirm SC-GPR as a chemoattractant receptor regulating cell localization. The homing function of SC-GPR occurs independently of CXCR-4 in the context within which it was tested in the Examples. While SC-GPR does not appear to depend on CXCR-4, the homing of cells to bone marrow in vivo may involve additional cell type-specific co-factors, which are present in vivo.

Successful approaches for hematopoietic stem cell manipulation will greatly facilitate the production of a large number of further differentiated precursor cells of a specific lineage, and in turn provide a larger number of differentiated hematopoietic cells for a wide variety of applications, including blood transfusions.

Thus, in some aspects a method for enhancing mobilization of hematopoietic cells in a subject is provided. The method involves administering to a subject a SC-GPR inhibitor to enhance mobilization of hematopoietic stem cells in the subject. As used herein a “SC-GPR inhibitor” is any compound which prevents the activity of a SC-GPR protein. SC-GPR inhibitors include but are not limited to SC-GPR binding agents which prevent SC-GPR activity e.g., anti-SC-GPR antibodies or fragments thereof; SC-GPR antagonists; SC-GPR blocking agents; SC-GPR antisense molecule and SC-GPR dominant negative proteins.

PCT application WO99/57245 (SmithKline Beecham Corporation) discloses methods of screening for agonists and antagonists of the interaction between the human KIAA0001 receptor and ligands thereof As mentioned above, the human KIAA0001 receptor has the same sequence as the human SC-GPR. One of ordinary skill in the art can identify SC-GPR antagonists using the methods described in PCT application WO99/57245.

The SC-GPR antagonists, SC-GPR blocking agents or other binding agents which prevent SC-GPR activity can be identified as described above for SC-GPR binding agents and then tested for effect on biological activity, using any of the assays described herein or otherwise known in the art. For instance, in vitro and in vivo assays for enhancing mobilization of hematopoietic stem cells is described in the Examples. These types of assays can be used to identify molecules that are SC-GPR inhibitors.

SC-GPR inhibitors also include antisense oligonucleotides that selectively bind to a plasma membrane SC-GPR nucleic acid molecule and dominant negative SC-GPR to reduce the expression of plasma membrane SC-GPR. Antisense oligonucleotides are useful, for example, for inhibiting SC-GPR expression in a cell in which it is ordinarily expressed.

As used herein, the term “antisense oligonucleotide” or “antisense” describes an oligonucleotide which hybridizes under physiological conditions to DNA comprising a particular gene or to an RNA transcript of that gene and, thereby, inhibits the transcription of that gene and/or the translation of the mRNA. The antisense molecules are designed so as to hybridize with the target gene or target gene product and thereby, interfere with transcription or translation of the target mammalian cell gene. Those skilled in the art will recognize that the exact length of the antisense oligonucleotide and its degree of complementarity with its target will depend upon the specific target selected, including the sequence of the target and the particular bases which comprise that sequence. The antisense must be a unique fragment. A unique fragment is one that is a ‘signature’ for the larger nucleic acid. It, for example, is long enough to assure that its precise sequence is not found in molecules outside of the SC-GPR gene. As will be recognized by those skilled in the art, the size of the unique fragment will depend upon its conservancy in the genetic code. Thus, some regions of the SC-GPR nucleic acids will require longer segments to be unique while others will require only short segments, typically between 12 and 32 base pairs (e.g. 12, 13, 14, 15, 16, 17, 18, 19, 20, 21, 22, 23, 24, 25, 26, 27, 28, 29, 30, 31 and 32 bases long).

It is preferred that the antisense oligonucleotide be constructed and arranged so as to bind selectively with the target under physiological conditions, i.e., to hybridize substantially more to the target sequence than to any other sequence in the target cell under physiological conditions. Based upon the known sequence of a gene that is targeted for inhibition by antisense hybridization, or upon allelic or homologous genomic and/or cDNA sequences, one of skill in the art can easily choose and synthesize any of a number of appropriate antisense molecules for use in accordance with the present invention. In order to be sufficiently selective and potent for inhibition, such antisense oligonucleotides should comprise at least 7 and, more preferably, at least 15 consecutive bases which are complementary to the target. Most preferably, the antisense oligonucleotides comprise a complementary sequence of 20-30 bases. Although oligonucleotides may be chosen which are antisense to any region of the gene or RNA (e.g., mRNA) transcripts, in preferred embodiments the antisense oligonucleotides are complementary to 5′ sites, such as translation initiation, transcription initiation or promoter sites, that are upstream of the gene that is targeted for inhibition by the antisense oligonucleotides. In addition, 3′-untranslated regions may be targeted. Furthermore, 5′ or 3′ enhancers may be targeted. Targeting to mRNA splice sites has also been used in the art but may be less preferred if alternative mRNA splicing occurs. In at least some embodiments, the antisense is targeted, preferably, to sites in which mRNA secondary structure is not expected (see, e.g., Sainio et al., Cell Mol. Neurobiol., (1994) 14(5):439-457) and at which proteins are not expected to bind. The selective binding of the antisense oligonucleotide to a mammalian target cell nucleic acid effectively decreases or eliminates the transcription or translation of the mammalian target cell nucleic acid molecule.

The invention also includes the use of a “dominant negative plasma membrane SC-GPR” polypeptide. A dominant negative polypeptide is an inactive variant of a protein, which, by interacting with the cellular machinery, displaces an active protein from its interaction with the cellular machinery or competes with the active protein, thereby reducing the effect of the active protein. For example, a dominant negative receptor which binds a ligand but does not transmit a signal in response to binding of the ligand can reduce the biological effect of expression of the ligand. Likewise, a dominant negative catalytically-inactive kinase which interacts normally with target proteins but does not phosphorylate the target proteins can reduce phosphorylation of the target proteins in response to a cellular signal. Similarly, a dominant negative transcription factor which binds to a promoter site in the control region of a gene but does not increase gene transcription can reduce the effect of a normal transcription factor by occupying promoter binding sites without increasing transcription.

The end result of the expression of a dominant negative polypeptide as used herein in a cell is a reduction in membrane expressed SC-GPR. One of ordinary skill in the art can assess the potential for a dominant negative variant of a protein, and using standard mutagenesis techniques to create one or more dominant negative variant polypeptides. For example, one of ordinary skill in the art can modify the sequence of the membrane SC-GPR by site-specific mutagenesis, scanning mutagenesis, partial gene deletion or truncation, and the like. See, e.g., U.S. Pat. No. 5,580,723 and Sambrook et al., Molecular Cloning: A Laboratory Manual, Second Edition, Cold Spring Harbor Laboratory Press, 1989. The skilled artisan then can test the population of mutagenized polypeptides for diminution in a selected and/or for retention of such an activity, or simply for presence in the plasma membrane. Other similar methods for creating and testing dominant negative variants of a protein will be apparent to one of ordinary skill in the art.

In one embodiment the subject is a bone marrow donor. By enhancing mobilization of bone marrow cells, the need for bone marrow isolation may be obviated. As a result of this mobilization, bone marrow cells leave the bone marrow and enter the blood circulation of the subject undergoing treatment. The circulating bone marrow cells can then be easily isolated using the techniques of the invention or other methods know in the art. For instance, these methods may reduce the need for large bone marrow donations for therapeutic procedures. The methods enable the isolation of hematopoietic stem cells from peripheral blood by encouraging localization from the bone marrow to the blood and thus, eliminating the need for bone marrow donation.

Hematopoietic stem cell manipulation is also useful as a supplemental treatment to chemotherapy, e.g., hematopoietic stem cells may be caused to localize into the peripheral blood and then isolated from a subject that will undergo chemotherapy, and after the therapy the cells can be returned. Thus, the subject in some embodiments is a subject undergoing or expecting to undergo an immune cell depleting treatment such as chemotherapy. Most chemotherapy agents used act by killing all cells going through cell division. Bone marrow is one of the most prolific tissues in the body and is therefore often the organ that is initially damaged by chemotherapy drugs. The result is that blood cell production is rapidly destroyed during chemotherapy treatment, and chemotherapy must be terminated to allow the hematopoietic system to replenish the blood cell supplies before a patient is re-treated with chemotherapy. This can be avoided using the methods of the invention.

Once the hematopoietic stem cells are mobilized from the bone marrow to the peripheral blood a blood sample can be isolated in order to obtain the hematopoietic stem cells. These cells can be transplanted immediately or they can be processed in vitro first. For instance, the cells can be expanded in vitro and/or they can be subjected to an isolation or enrichment procedure. It will be apparent to those of ordinary skill in the art that the crude or unfractionated blood products can be enriched for cells having “hematopoietic stem cell” characteristics using the methods of the invention for detecting SC-GPR such as those described above or in a number of other ways. Some of the other ways include, e.g., depleting the blood product from the more differentiated progeny. The more mature, differentiated cells can be selected against, via cell surface molecules they express other tha SC-GPR. Additionally, the blood product can be fractionated selecting for CD34⁺ cells. Such selection can be accomplished using, for example, commercially available magnetic anti-CD34 beads (Dynal, Lake Success, N.Y.). In preferred embodiments, however, the methods of the invention may be used to isolate the hematopoietic stem cells.

The invention also encompasses methods for modulating hematopoietic cell function by manipulating SC-GPR expression in such cells. Such methods are useful, for example, for improving the efficiency of targeting transplanted hematopoietic cells to bone marrow (e.g., by transducing a SC-GPR nucleic acid into the hematopoietic cells to be transplanted). SC-GPR transduction may also be used to confer true stem cell characteristics to more mature progenitor cell progeny (e.g., by transducing a SC-GPR nucleic acid into a progenitor cell).

Thus, hematopoietic stem cells may be modulated to encourage their migration to bone marrow. This is accomplished using a SC-GPR activator. An “SC-GPR activator” as used herein refers to any compound that increases SC-GPR activity, either by directly or indirectly activating endogenous SC-GPR or by inducing expression of exogenous SC-GPR protein within the cell.

Compounds that directly or indirectly activate SC-GPR include binding agents and other compounds that function as agonists of SC-GPR. WO99/57245 teaches that UDP-glucose, and some closely related molecules, potently activate the KIAA0001 receptor. A recent article also identified UDP conjugated carbohydrates as activators for this receptor (Chambers et al., 2000 J Biol Chem 275, 10767-71.). Specifically, UDP-glucose, UDP-glucuronic acid, UDP-galactose and UDP-N-acetylglucosamine were noted to bind to the receptor and induce calcium flux. While UDP-glucans may interact with SC-GPR, these compounds are generally regarded as intracellular metabolic intermediates and are unlikely to be the sole ligands for this cell surface receptor. N-acetylglucosamine, which is a SC-GPR activator, has been used to elute lectins from stem cells. Other agonists can be identified by those of skill in the art using the assays described herein as well as others known in the art.

One type of SC-GPR activator that functions by inducing expression of exogenous SC-GPR protein within the cell is a nucleic acid vector expressing SC-GPR. A SC-GPR nucleic acid can be delivered to a cell such that the SC-GPR peptide will be expressed in the membrane of the cell. The SC-GPR expression vectors and other relevant expression vectors described herein can be prepared and inserted into cells using routine procedures known in the art. These procedures are set forth below in more detail. A “SC-GPR nucleic acid”, as used herein, refers to a nucleic acid molecule which: (1) hybridizes under stringent conditions to a nucleic acid having the sequence of SEQ ID NO: 10 and (2) codes for a SC-GPR polypeptide or functionally active fragments thereof. The preferred SC-GPR nucleic acid has the nucleic acid sequence of SEQ ID NO: 10 (the nucleic acid encoding the human SC-GPR polypeptide). The SC-GPR nucleic acids may be intact SC-GPR nucleic acids which include the nucleic acid sequence of SEQ ID No.: 10 as well as homologs and alleles of a nucleic acid having the sequence of SEQ ID NO: 10. Intact SC-GPR nucleic acids further embrace nucleic acid molecules which differ from the sequence of SEQ ID NO: 10 in codon sequence due to the degeneracy of the genetic code. The SC-GPR nucleic acids of the invention may also be functionally equivalent variants, analogs and fragments of the foregoing nucleic acids. “Functionally equivalent”, in reference to a SC-GPR nucleic acid variant, analog or fragment, refers to a nucleic acid that codes for a SC-GPR polypeptide that is capable of functioning as a SC-GPR

Another type of compound which increases the expression of exogenous SC-GPR in a cell is an exogenously added SC-GPR protein or functionally active fragment thereof which is targeted to the membrane. A “SC-GPR protein or peptide”, as used herein, refers to a protein or peptide having an amino acid sequence of SEQ ID NO: 1, 2, or 3 or functional fragments or variants thereof. Optionally the SC-GPR peptide may be conjugated to a plasma membrane targeting domain.

There are many ways to induce expression of SC-GPR in a plasma membrane of a cell. For instance, it is possible to insert an intact SC-GPR, or functional fragment thereof, into a plasma membrane using delivery vehicles such as liposomes. SC-GPR is a naturally occurring membrane protein having several transmembrane spanning regions including many hydrophobic residues. Proteins of this type ca Spontaneously insert into a biological membrane in an aqueous environment. See, e.g., U.S. Pat. No. 5,739,273 (which is hereby incorporated by reference) describing properties of bacteriorhodopsin C helix, a transmembrane spanning protein. The SC-GPR can be inserted in to a biological membrane consistent with the methods described in U.S. Pat. No. 5,739,273 for inserting bacteriorhodopsin C into a membrane, including in lipid vesicles and by modification of various residues to increase the hydrophobicity of the molecule, without altering the function. Additionally SC-GPR can be conjugated to a molecule which will insert in the membrane, causing the SC-GPR to also insert in the membrane.

As set forth in U.S. Pat. No. 5,739,273 cell membranes are composed mainly of phospholipids and proteins, both containing hydrophobic and hydrophilic groups. The lipids orient themselves into an orderly bilayer configuration within the membrane core with the hydrophobic chains facing toward the center of the membrane while the hydrophilic portions are oriented toward the outer and inner membrane surfaces. The proteins are dispersed throughout the lipid layer, in some instances protruding through the surface of the membrane or extending from one side of the membrane to the other with some of the hydrophobic residues being buried in the interior of the lipid bilayer.

U.S. Pat. No. 5,739,273 teaches that a synthetic polypeptide maintaining the characteristics of a native polypeptide by including a hydrophobic alpha-helical transmembrane region containing one or more acidic or basic amino acids can be generated. Preferably, the amino acids are aspartic acid, glutamic acid, lysine, arginine or histidine. This is based on the teachings of Popot and Engelman, Biochem. 29:4031-4037 (1990), that recently proposed a two-stage model of helix formation for transmembrane proteins in which the alpha-helices first insert into the lipid bilayer and then assemble into a tertiary structure that includes interactions with other intramembrane alpha-helices of the protein or with alpha-helices of other polypeptides in the membrane.

The SC-GPR insertion into the membrane can be enhanced using lipid vesicles. Lipid vesicles such as micelles can be formed by the addition of phospholipids to achieve a specific ratio of protein to phospholipid. The orientation of the chimeric protein components of the micelles can be controlled also, so that the micelles have an outer surface which is predominantly composed of the phospholipid moieties or predominantly composed of the protein moieties. The size of the micelles may also be controlled by varying the detergent employed, the nature of the added phospholipid, or the phospholipid/protein ratio.

Generally, the size of liposomes directly affects the rate at which they are cleared from the bloodstream. For example, smaller liposomes and negatively charged liposomes appear to be more stable and accumulate in the spleen and liver. Thus, the micelles and liposomes can be tailored to remain in the bloodstream for a desired period and to be delivered to specific organs. For example, small micelles can be formed with an outer surface exhibiting a predominantly negative charge from the phosphoinositol moiety.

SC-GPR proteins include the intact native SC-GPR in an isolated form as well as functionally active fragments and variants thereof. The native SC-GPR protein has an amino acid sequence as presented in SEQ ID NO: 1, 2, or 3 (human, mouse, and rat respectively). A targeting moiety may optionally be coupled to the SC-GPR peptide, particularly if the peptide is a fragment of the SC-GPR, which may not be capable of spontaneously inserting into the membrane. The molecules may be directly coupled to one another, such as by conjugation or may be indirectly coupled to one another where, for example, the targeting moiety is on the surface of a liposome and the SC-GPR peptide is contained within the liposome. If the molecules are linked to one another, then the targeting moiety is covalently or noncovalently bound to the SC-GPR peptide in a manner that preserves the targeting specificity of the targeting moiety. As used herein, “linked” or “linkage” means two entities are bound to one another by any physiochemical means. It is important that the linkage be of such a nature that it does not impair substantially the effectiveness of the SC-GPR peptide or the binding specificity of the targeting moiety. Keeping these parameters in mind, any linkage known to those of ordinary skill in the art may be employed, covalent or noncovalent. Such means and methods of linkage are well known to those of ordinary skill in the art.

Linkage according to the invention need not be direct linkage. The components of the compositions of the invention may be provided with functionalized groups to facilitate their linkage and/or linker groups may be interposed between the components of these compositions to facilitate their linkage. In addition, the components of the present invention may be synthesized in a single process, whereby the components could be regarded as one in the same entity.

Specific examples of covalent bonds include those wherein bifunctional cross-linker molecules are used. The cross-linker molecules may be homobifunctional or heterobifunctional, depending upon the nature of the molecules to be conjugated. Homobifunctional cross-linkers have two identical reactive groups. Heterobifunctional cross-linkers have two different reactive groups that allow sequential conjugation reaction. Various types of commercially available cross-linkers are reactive with one or more of the following groups: primary amines, secondary amines, sulfhydriles, carboxyls, carbonyls and carbohydrates.

Non-covalent methods of conjugation also may be used to join the targeting moiety and the SC-GPR peptide. Non-covalent conjugation may be accomplished by direct or indirect means including hydrophobic interaction, ionic interaction, intercalation, binding to major or minor grooves of a nucleic acid and other affinity interactions.

Covalent linkages may be noncleavable in physiological environments or cleavable in physiological environments, such as linkers containing disulfide bonds. Such molecules may resist degradation and/or may be subject to different intracellular transport mechanisms. One of ordinary skill in the art will be able to ascertain without undue experimentation the preferred bond for linking the targeting moiety and the SC-GPR inhibitor or activator, based on the chemical properties of the molecules being linked and the preferred characteristics of the bond.

For indirect linkage, the targeting moiety may be part of a particle, such as a liposome, which targets the liposome to the hematopoietic cell. The liposome, in turn, may contain the SC-GPR activator. The manufacture of liposomes containing SC-GPR activator is fully described in the literature. Many are based upon cholesteric molecules as starting ingredients and/or phospholipids. They may be synthetically derived or isolated from natural membrane components. Virtually any hydrophobic substance can be used, including cholesteric molecules, phospholipids and fatty acids preferably of medium chain length (12C-20C). Preferred are naturally occurring fatty acids of between 14 and 18 carbons in length. These molecules can be attached to the SC-GPR activator with the lipophilic anchor inserting into the membrane of a liposome and the SC-GPR activator tethered on the surface of the liposome for targeting the liposome to the cell.

When a functionally active peptide fragment of the SC-GPR is used rather than the intact SC-GPR, it may be desirable to attach the fragment to a plasma membrane targeting sequence to ensure that it is delivered to the plasma membrane. Plasma membrane targeting sequences include hydrophobic moieties and membrane attachment domains. Hydrophobic moieties are well known in the art. A “membrane attachment domain,” as used herein, refers to a domain that spans the width of a cell/plasma membrane, or any part thereof, and that functions to attach a SC-GPR activator to a cell membrane. The amino acid sequences of exemplary membrane attachment domains are described in Pigott and Power, The adhesion Molecule Facts Book San Diego: Academic Press, Inc. (1993) and Barclay et al., The Leukocyte Antigen Facts Book San Diego: Academic Press, Inc. (1993), each of which is incorporated herein by reference.

The term “heterologous,” as used herein in reference to a membrane attachment domain operatively fused to a SC-GPR peptide, means a membrane attachment domain derived from a source other than the gene encoding the SC-GPR. A heterologous membrane attachment domain can be synthetic or can be encoded by a gene distinct from the gene encoding the SC-GPR to which it is fused.

The term “operatively fused,” as used herein in reference to a SC-GPR peptide and a heterologous membrane attachment domain, means that the SC-GPR peptide and membrane attachment domain are fused in the correct reading frame such that, under appropriate conditions, a full-length fusion protein is expressed. One skilled in the art would recognize that such a fusion protein can comprise, for example, an amino-terminal SC-GPR peptide operatively fused to a carboxyl-terminal heterologous membrane attachment domain or can comprise an amino-terminal heterologous membrane attachment domain operatively fused to a carboxyl-terminal SC-GPR peptide.

The term “membrane-bound,” as used herein in reference to a fusion protein means stably attached to a cellular membrane. The term “fusion protein,” as used herein, means a hybrid protein including a synthetic or heterologous amino acid sequence.

The invention in one aspect is a method for supplementing bone marrow by administering to a subject a SC-GPR enriched hematopoietic cell population to supplement the bone marrow of the subject. An “SC-GPR enriched hematopoietic cell population” as used herein is one which has a high proportion of cells expressing active SC-GPR. In one embodiment at least 50% of the cells express SC-GPR One method for generating this population is to use an activator, e.g., transduce expression of SC-GPR. Another method is to selectively isolate and administer a hematopoietic cell population which expresses SC-GPR.

The invention also includes SC-GPR-transduced hematopoietic cells. A “SC-GPR transduced hematopoietic cell” as used herein is a cell of hematopoietic origin which expresses a SC-GPR molecule as a result of exogenous genetic material. In one embodiment the SC-GPR-transduced hematopoietic cell is prepared by transduction of a cell of hematopoietic origin with a nucleic acid of SEQ ID No. 10.

As shown in the Examples, transduced cell lines did not alter their cell cycling, but forced ectopic expression of SC-GPR in primary cord blood CD34+ CD38− cells led to reduced colony output and sustained colony production in the context of a stromal ligand source. SC-GPR therefore appears to inhibit either proliferative or differentiation kinetics in primary hematopoietic progenitor cells. Chemokines have been previously associated with both transmigratory and inhibitory roles, notably SLC in hematopoietic progenitors acting via the CXCR3 or CCR7 receptors (Kim and Broxmeyer, 1999. J Leukoc Biol 66, 455-61.). Since that chemokine may activate two receptors, it has not been definitively demonstrated that the same receptor ca Signal both inhibitory and migratory signals. SC-GPR may therefore be unique in that capacity. Transducing the cord blood CD34+ cells required manipulation which resulted in exhaustion of the CAFC and LTC-IC phenotype with the notable exception of those cells expressing the SC-GPR receptor. The data suggest that expression and activation of SC-GPR may induce or retain a more primitive phenotype of the expressing cells.

As used herein, “transduction of hematopoietic cells” refers to the process of transferring exogenous genetic material into a cell of hematopoietic origin. The terms “transduction”, “transfection” and “transformation” are used interchangeably and refer to the process of transferring exogenous genetic material into a cell. As used herein, “exogenous genetic material” refers to nucleic acids or oligonucleotides, either natural or synthetic, that are introduced into the hematopoietic stem cells. The exogenous genetic material may be a copy of that which is naturally present in the cells, or it may not be naturally found in the cells. It typically is at least a portion of a naturally occurring gene which has been placed under operable control of a promoter in a vector construct. In preferred embodiments the hematopoietic stem cells are transduced with SC-GPR.

The invention also embraces methods for in vitro culture of hematopoietic cells to produce differentiated cells of non-hematopoietic lineage. Such methods involve contacting a hematopoietic cell with SC-GPR (e.g., by transduction to produce SC-GPR-transduced hematopoietic cell) under conditions sufficient to confer hematopoietic stem cell characteristics (or properties) to the hematopoietic cell, and culturing the hematopoietic cell having stem cell characteristics in an environment that promotes hematopoietic cell differentiation, under conditions and for a period of time to produce differentiated cells of non-hematopoietic lineage.

In some embodiments, the environment comprises factors that direct differentiation of hematopoietic cells to produce differentiated cells of non-hematopoietic lineage selected from the group consisting of mesenchymal, parenchymal, neuronal, endothelial, and epithelial cells. In a certain embodiment, the hematopoietic cells are SC-GPR-negative cells that are SC-GPR activated (e.g, transduced) to acquire stem cell characteristics, and the environment comprises growth factors selected from the group consisting of bFGF and TGF-β, to produce mesenchymal cells. In a further embodiment, the hematopoietic cells are SC-GPR-negative cells that are SC-GPR activated (e.g, transduced) to acquire stem cell characteristics, and the environment comprises growth factors selected from the group consisting of putrescine, progesterone, sodium selenite, insulin, transferrin, EGF, NGF, and bFGF, to produce neuronal cels. In a yet further embodiment, the hematopoietic cells are SC-GPR-negative cells that are SC-GPR activated (e.g, transduced) to acquire stem cell characteristics, and the environment comprises growth factors selected from the group consisting of IL-3, SCF, TGF-β1, and Flk-2/Flt-3 ligand, to produce epithelial cells. In a yet further embodiment, the hematopoietic cells are SC-GPR-negative cells that are SC-GPR activated (e.g, transduced) to acquire stem cell characteristics, and the environment comprises VEGF, to produce endothelial cells. In a still further embodiment the hematopoietic progenitor cells are CD34⁺ and/or CD34⁻, and the environment comprises EGF, bFGF, and SF/HGF, to produce parenchymal cells.

Various techniques may be employed for introducing nucleic acids into cells. Such techniques include transfection of nucleic acid-CaPO₄ precipitates, transfection of nucleic acids associated with DEAE, transfection with a retrovirus including the nucleic acid of interest, liposome mediated transfection, and the like. For certain uses, it is preferred to target the nucleic acid to particular cells. In such instances, a vehicle used for delivering a nucleic acid according to the invention into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. For example, where liposomes are employed to deliver the nucleic acids, proteins which bind to a surface membrane protein associated with endocytosis may be incorporated into the liposome formulation for targeting and/or to facilitate uptake. Such proteins include proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

In the present invention, the preferred method of introducing exogenous genetic material into hematopoietic cells is by transducing the cells in vitro using replication deficient retroviruses. Replication-deficient retroviruses are capable of directing synthesis of all virion proteins, but are incapable of making infectious particles. Accordingly, these genetically altered retroviral vectors have general utility for high-efficiency transduction of genes in cultured cells, and specific utility for use in the method of the present invention. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in the art.

The major advantage of using retroviruses is that the viruses insert efficiently a single copy of the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types. The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome. Despite these apparent limitations, delivery of a therapeutically effective amount of a gene such as SC-GPR via a retrovirus can be efficacious if the efficiency of transduction is high and/or the number of target cells available for transduction is high.

Yet another viral candidate useful as an expression vector for transformation of hematopoietic cells is the adenovirus, a double-stranded DNA virus. Like the retrovirus, the adenovirus genome is adaptable for use as an expression vector for gene transduction, i.e., by removing the genetic information that controls production of the virus itself. Because the adenovirus functions usually in an extrachromosomal fashion, the recombinant adenovirus does not have the theoretical problem of insertional mutagenesis. On the other hand, adenoviral transformation of a target hematopoietic cell may not result in stable transduction. However, more recently it has been reported that certain adenoviral sequences confer intrachromosomal integration specificity to carrier sequences, and thus result in a stable transduction of the exogenous genetic material.

Thus, as will be apparent to one of ordinary skill in the art, a variety of suitable vectors are available for transferring exogenous genetic material into hematopoietic cells. The selection of an appropriate vector to deliver a the gene and the optimization of the conditions for insertion of the selected expression vector into the cell, are within the scope of one of ordinary skill in the art without the need for undue experimentation. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any nontranslated DNA sequence which works contiguous with the coding sequence (in cis) to change the basal transcription level dictated by the promoter. Preferably, the exogenous genetic material is introduced into the hematopoietic cell genome immediately downstream from the promoter so that the promoter and coding sequence are operatively linked so as to permit transcription of the coding sequence. A preferred retroviral expression vector includes an exogenous promoter element to control transcription of the inserted exogenous gene. Such exogenous promoters include both constitutive and inducible promoters.

Naturally-occuring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88:4626-4630 (1991)), adenosine deaminase, phosphoglycerol kinase (PGK), pyruvate kinase, phosphoglycerol mutase, the actin promoter (Lai et al., Proc. Natl. Acad. Sci. USA 86: 10006-10010 (1989)), and other constitutive promoters known to those of skill in the art. In addition, many viral promoters function constitutively in eucaryotic cells. These include: the early and late promoters of SV40; the long terminal repeats (LTRS) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a gene, i.e., SC-GPR in the genetically modified hematopoietic cell. Selection and optimization of these factors for expression of an effective quantity of the gene is deemed to be within the scope of one of ordinary skill in the art without undue experimentation, taking into account the above-disclosed factors.

In addition to at least one promoter and at least one heterologous nucleic acid, the expression vector optionally includes a selection gene, for example, a neomycin resistance gene, for facilitating selection of hematopoietic cells that have been transfected or transduced with the expression vector. Alternatively, the hematopoietic cells are transfected with two or more expression vectors, at least one vector containing the gene(s), the other vector containing a selection gene. The selection of a suitable promoter, enhancer, selection gene and/or signal sequence (described below) is deemed to be within the scope of one of ordinary skill in the art without undue experimentation.

The selection and optimization of a particular expression vector for expressing a specific gene product in an isolated hematopoietic cell is accomplished by obtaining the gene, preferably with one or more appropriate control regions (e.g., promoter, insertion sequence); preparing a vector construct comprising the vector into which is inserted the gene; transfecting or transducing cultured hematopoietic cells in vitro with the vector construct; and determining whether the gene product is present in the cultured cells.

Gene therapy is a rapidly growing field in medicine with an enormous clinical potential. Traditionally, gene therapy has been defined as a procedure in which an exogenous gene is introduced into the cells of a patient in order to correct an inborn genetic error. It may be accomplished using ex vivo or in vivo methods. Research in gene therapy has been ongoing for several years in several types of cells in vitro and in animal studies, and more recently a number of clinical trials have been initiated. The human hematopoietic system is an ideal choice for gene therapy in that hematopoietic stem cells are readily accessible for treatment, particularly in combination with the methods of the invention, and they are believed to possess a limited self-renewal capabilities (incurring lifetime therapy), and upon re-infusion, can expand and repopulate the marrow.

The methods of the invention can provide improvements in these procedures. Thus, in some embodiments the hematopoietic cells identified or isolated according to the invention can be further manipulated for use in gene therapy applications. The gene can be added to the cells by any of the methods described above or any other methods known in the art. In some embodiments gene therapy is accomplished using hematopoietic stem cells identified or isolated using the methods of the invention and transduced with a therapeutic gene. In other embodiments the gene therapy is accomplished using a hematopoietic stem cell population transduced with both SC-GPR and a gene expressing a therapeutic, such that at least some of the cells are transduced with SC-GPR and/or at least some others are transduced with a therapeutic gene.

Methods for expressing exogenous genes in vitro, ex vivo, and in vivo are well known in the art and abbreviated methods are described herein. An “ex vivo” method as used herein is a method which involves isolation of a cell from a subject, manipulation of the cell outside of the body, and reimplantation of the manipulated cell into the subject. The ex vivo procedure may be used on autologous or heterologous cells. Table 1 provides a summary of human gene therapy protocols approved by RAC from 1990-1994. In some embodiments the gene therapy methods of the invention encompass the use of the genes listed in Table 1. Other gene therapies are known in the art, and are also encompassed by the methods of the invention.

TABLE 1
Human Gene Therapy Protocols Approved by RAC: 1990-1994

| | | |
|---|---|---|
| Severe combined | Autologous lymphocytes transduced | Jul. 31, 1990 |
| Immune | with human ADA gene | |
| deficiency | | |
| (SCID) due to | | |
| adenosine | | |
| deaminase | | |
| (ADA) deficiency | | |
| Advanced cancer | Tumor-infiltrating lymphocytes | Jul. 31, 1990 |
| | transduced with tumor necrosis | |
| | factor gene | |
| Advanced cancer | Immunization with autologous cancer | Oct. 7, 1991 |
| | cells transduced with tumor necrosis | |
| | factor gene | |
| Advanced cancer | Immunization with autologous cancer | Oct. 7, 1991 |
| | cells transduced with interleukin-2 | |
| | gene | |
| Asymptomatic | Murine Retro viral vector encoding | Jun. 7, 1993 |
| patients infected | HIV-1 genes [HIV-IT(V)] | |
| with HIV-1 | | |
| AIDS | Effects of a transdominant form | Jun. 7, 1993 |
| | of rev gene on AIDS intervention | |
| Advanced cancer | Human multiple-drug resistance | Jun. 8, 1993 |
| | (MDR) gene transfer | |
| HIV infection | Autologous lymphocytes transduced | Sep. 10, 1993 |
| | with catalytic ribozyme that cleaves | |
| | HIV-1 RNA (Phase I study). | |
| Metastatic | Genetically engineered autologous | Sep. 10, 1993 |
| melanoma | tumor vaccines producing | |
| | interleukin-2 | |
| HIV infection | Murine Retro viral vector encoding | Dec. 3, 1993 |
| | HIV-IT (V) genes (open label | |
| | Phase I/II trial) | |
| HIV infection | Adoptive transfer of syngeneic | Mar. 3, 1994 |
| (identical twins) | cytotoxic T lymphocytes | |
| | (Phase I/II pilot study) | |
| Breast cancer | Use of modified Retro virus to | Jun. 9, 1994 |
| (chemoprotection | introduce chemotherapy resistance | |
| during therapy) | sequences into normal hematopoietic | |
| | cells (pilot study) | |
| Fanconi's anemia | Retro viral mediated gene transfer of | Jun. 9, 1994 |
| | Fanconi anemia complementation | |
| | group C gene to hematopoietic | |
| | progenitors | |
| Metastatic | Autologous human granulocyte | ORDA/ |
| prostate | macrophage-colony stimulating factor | NIH |
| carcinoma | gene transduced prostate cancer | Aug. 3, 1994* |
| | vaccine *(first protocol to be | |
| | approved under the accelerated | |
| | review process; ORDA = Office of | |
| | Recombinate DNA Activities) | |
| Metastatic | In vivo infection with breast-targeted | Sep. 12, 1994 |
| breast cancer | Retro viral vector expressing anti- | |
| | sense c-fox or antisense c-myc RNA | |
| Metastatic | Non-viral system (liposome-based) | Sep. 12, 1994 |
| breast cancer | for delivering human interleukin-2 | |
| (refractory | gene into autologous tumor cells | |
| or recurrent) | (pilot study) | |
| Mild Hunter | Retro viral-mediated transfer of the | Sep. 13, 1994 |
| syndrome (muco- | iduronate-2-sulfatase gene into | |
| polysaccharidosis | lymphocytes | |
| type II) | | |
| Advanced | Use of recombinant adenovirus | Sep. 13, 1994 |
| mesothelioma | (Phase I study) | |

The foregoing represent only examples of genes that can be delivered according to the methods of the invention. Suitable promoters, enhancers, vectors, etc., for such genes are published in the literature associated with the foregoing trials. In general, useful genes replace or supplement function, including genes encoding missing enzymes such as adenosine deaminase (ADA) which has been used in clinical trials to treat ADA deficiency and cofactors such as insulin and coagulation factor VIII. Genes which affect regulation can also be administered, alone or in combination with a gene supplementing or replacing a specific function. For example, a gene encoding a protein which suppresses expression of a particular protein-encoding gene can be administered. The invention is particularly usefull in delivering genes which stimulate the immune response, including genes encoding viral antigens, tumor antigens, cytokines (e.g. tumor necrosis factor) and inducers of cytokines (e.g. endotoxin).

A “subject” as used herein refers to a human or non-human mammal including but not limited to primates, dogs, cats, horses, sheep, goats, cows, rabbits, pigs and rodents.

When the cells or any compounds (referred to as therapeutic compositions) are administered to a subject, the therapeutic compositions may be administered in pharmaceutically acceptable preparations. Such preparations may routinely contain pharmaceutically acceptable concentrations of salt, buffering agents, preservatives, compatible carriers, and optionally other therapeutic agents.

The therapeutic composition may be administered by any conventional route, including injection or by gradual infusion over time. The administration may, depending on the composition being administered, for example, be oral, pulmonary, intravenous, intraperitoneal, intramuscular, intracavity, subcutaneous, or transdermal. Techniques for preparing aerosol delivery systems containing active agents are well known to those of skill in the art. Generally, such systems should utilize components which will not significantly impair the biological properties of the active agents (see, for example, Sciarra and Cutie, “Aerosols,” in Remington's Pharmaceutical Sciences 18th edition, 1990, pp 1694-1712; incorporated by reference). Those of skill in the art can readily determine the various parameters and conditions for producing aerosols without resort to undue experimentation. When using antisense preparations, intravenous or oral administration are preferred.

The compositions are administered in effective amounts. An “effective amount” is that amount of a composition that alone, or together with further doses, produces the desired response, e.g. increases or decreases expression or activity of SC-GPR or, for cells, results in an increase in hematopoeitic stem cells in the bone marrow. The term “therapeutic composition” is used synonymously with the terms “active compound”, “active agent” or active composition” and as used herein refers to any of the active compounds of the invention which produce a biological effect, e.g., SC-GPR activators, inhibitors, SC-GPR transduced cells, enriched hematopoietic stem cell preparations, etc. In the case of treating a particular disease or condition characterized by immune deficiency, the desired response is any improvement in immune system function. This may involve only an increase in the actual numbers of hematopoietic stem cell, slowing of onset or progression of an infectious disease arising from the immune system dysfunction, temporarily, although more preferably, it is involves an actual improvement in the prevention of disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of therapeutic composition for producing the desired response in a unit of weight or volume suitable for administration to a patient. The response can, for example, be measured by determining the effect on cell mobilization following administration of the therapeutic composition via a reporter system, or by isolating cells and measuring mobility in vitro. Other assays will be known to one of ordinary skill in the art and can be employed for measuring the level of the response.

The doses of the active compounds administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, doses of a therapeutic composition, other than cells, are formulated and administered in doses between 1 ng and 1 mg, and preferably between 10 ng and 100 μg, according to any standard procedure in the art. Where nucleic acids encoding a SC-GPR protein or variants thereof are employed, doses of between 1 ng and 0.1 mg generally will be formulated and administered according to standard procedures. Other protocols for the administration of therapeutic compositions will be known to one of ordinary skill in the art, in which the dose amount, schedule of injections, sites of injections, mode of administration and the like vary from the foregoing. Administration of therapeutic compositions to mammals other than humans, e.g. for testing purposes or veterinary therapeutic purposes, is carried out under substantially the same conditions as described above.

The following description of experiments performed is exemplary and non-limiting to the scope of the claimed invention.

### EXAMPLES

Hematopoietic stem cells undergo a development stage-specific translocation during ontogeny and ultimately reside in the adult bone marrow. Maintenance of this highly regenerative cell pool through adult life is dependent upon the relative quiescence of stem cells. The following examples demonstrate new methods for identifying and manipulating hematopoeitic stem cells for improved therapeutic purposes.

We generated cDNA from quiescent human hematopoietic stem-like cells derived from bone marrow and identified by subtractive cloning a seven transmembrane molecule with a signature motif of the chemokine receptor family. Antiserum raised against this gene product identified cells from human fetal bone marrow, but not other fetal hematopoietic organs and very rare cells from adult bone marrow. These cells were enriched for quiescent cells with the ability to sustain mature blood cell generation for prolonged periods on stromal feeder layers. Receptor activation was induced by bone marrow stroma specifically and cells expressing the receptor transmigrated toward bone marrow stroma in vitro and homed to bone marrow in vivo. Transduction of primary CD34+ cells altered their functional properties resulting in enhanced activity in stem cell assays. Stem cell-G protein-coupled receptor-1 (SC-GPR-1) is a chemokine receptor that identifies quiescent bone marrow-derived hematopoietic stem cells, participates in stem cell homing to bone marrow and alters stem cell differentiation kinetics. Thus, the expression and activity of this molecule can be manipulated to enhance therapeutic procedures.

### Example 1

Cloning of a Differentially Expressed Gene Encoding a Transmembrane Protein.

Methods:

Construction and Screening of a Subtracted cDNA Library.

Human bone marrow cells were obtained from healthy volunteers who provided written informed consent to a protocol approved by the Massachusetts General Hospital Institutional Review Board (IRB). Cord blood was obtained from St. Louis University using IRB approved protocols. CD34+ cells were isolated with MACS (Miltenyi, Bergisch Gladbach, Germany) according to the manufacture's instruction. Quiescent CD34+CD38− cells were prepared as described except using a higher concentration of 5-FU (Pharmacia Inc, Kalamazoo, Mich.) (Berardi, A. C., et al., (1995) Science 267, 104-8.) Briefly, CD34+38− cells were incubated at 37C with 5% CO2 in IMDM (GibcoBRL, Grand Island, N.Y.) containing 10% fetal calf serum (Sigma, ST. Louis, Mo.) supplemented with KL (100 mg/ml) and IL-3 (100 ng/ml) with 5-FU (2.5 mg/ml). Approximately 10-20 cells were picked by Quixell micromanipulator (Stoelting Co., Wood Dale, Ill.) and transferred to a PCR tube containing lysis/binding buffer (100 mM Tris-HCl, PH8.0, 500 mM LiCL, 10 mM EDTA, 1% LIDS, 5 mM DTT). The mRNA was purified by adding 10 ul of oligo(dT)-linked magnetic beads (Dynal A. S., Oslo, Norway). After 10 min incubation at room temperature, the magnetic beads were washed three times with 50 ul of RT buffer. RT-PCR was carried out in situ as described (Brady, G., et al. (1995) [published erratum appears in Curr Biol 1995 Oct 1;5(10):1201]. Curr Biol 5, 909-22.) Further amplification of cDNA and subtractions were carried out as described (Karrer, E. et al. (1995) Proc Natl Acad Sci USA 92, 3814-8.) Briefly, cDNA was digested completely with Alu I and Alu I plus Rsa I separately to yield 200-600 bp cDNA fragments which prevents disproportionate PCR amplification of smaller cDNAs. After seven rounds of subtractive hybridization against biotinylated cDNA generated from CD34+ CD38+ cells, subtracted cDNAs were cloned into the vector Topo2.1. DNA (Invitrogen, Carlsbad, Calif.). A mouse clone was identified from NCBI dbest database. Blast searches of the dbest data base with a conserved region between SC-GPR and VTR 15-20 retrieved an EST clone, (GenBank accession number; AA139729). The clone generated by the IMAGE consortium was obtained from ATCC as a EcoRI-NotI insert in the pT7T3D-Pac vector (Pharmacia, Piscataway, N.J.), and full-length clone was identified and sequenced subsequently.

Antibody Preparation

An anti-SC-GPR antibody was generated using a peptide (named N-SC-GPR, MINSTSTQPPDESCSQN (SEQ ID NO. 4)) which spans 17 amino acids of first extracellular domain of SC-GPR protein. N-SC-GPR was conjugated with a carrier protein, Keyhole Lymphet Hemocyanin (KLH), and injected into rabbits. After the second boost, the rabbits were bled, and the serum was isolated and used for affinity purification. SC-GPR peptide was immobilized to SulfoLink Coupling Gel (Pierce, Rockford, Ill.). Antiserum was mixed with SC-GPR peptide conjugated affinity gels and gently rocked for 1 hr at room temperature. After extensive washing with PBS, antibody was eluted with 3M KSCN and used for FACS analysis and immunoprecipitation.

Western Analysis and Immunoprecipitation

Coupled in vitro transcription/translation was carried out using the TNT-coupled reticulocyte lysate system (Promega, Madison, Wis.) according to the manufacturer's instructions. The lysate was mixed with affinity purified anti-SC-GPR antibody and incubated at 4° C. overnight. The immunoprecipitates were washed three times with RIPA buffer (150 mM NaCl, 1% NP-40, 0.5% DOC, 0.1% SDS, 50 mM Tris, PH 8.0) and boiled for 5 min in 30 ul of sample buffer. 20 ul was loaded into a Laemmli 10% SDS-polyacrylamide gel. The blot was probed with anti-HA monoclonal antibody using the ECL detection system (Amersham, Buckinghamshire, England). To confirm the results, the experiment was repeated using anti-HA antibody for immunoprecipitation and anti-SC-GPR antibody for Western analysis. Both of the methods detected protein of similar molecular weight.

Results:

We used our strategy of anti-metabolite treatment combined with cytokines known to drive progenitor populations into active cycle to selectively eliminate actively cycling cells (Berardi et al., 1995 Science 267, 104-8.). This directed suicide method exploits the known cytokine unresponsiveness of the stem cell compartment (Traycoff et al., 1996 Exp Hematol 24, 299-306.; Traycoff et al., 1995 Blood 85, 2059-68.; Veena et al., 1998 Blood 91, 3693-701.) and yields a rare population of human bone marrow derived cells (1 in 10⁶ mononuclear cells) which are small in size, quiescent and only produce progeny after prolonged exposure to bone marrow stroma. A similar strategy has been used by others to isolate cells shown to have efficient in vitro and in vivo stem cell-like characteristics including repopulation of NOD/SCID mice (Bertolini et al., 1997 Blood 90, 3027-36.; Bertolini et al., 1997, Exp Hematol 25, 350-6.). The rarity of these cells has required the use of PCR-based strategies for characterization of the gene expression profile of the cells Berardi et al., 1995 Science 267, 104-8; Cheng et al., 1996 Proc Natl Acad Sci USA 93, 13158-63.). Based on analysis of individual cells isolated by micropipette, the population of cells isolated by the directed suicide technique has been shown to be molecularly homogeneous (Berardi et al., 1995 Science 267, 104-8; Cheng et al., 1996 Proc Natl Acad Sci USA 93, 13158-63.).

In an effort to use the cell population as a reagent for identification of novel regulatory genes, we generated cDNA from ˜20 of these cells using a polydT primed RT PCR technique that has been shown to preserve fidelity in both complexity and relative abundance of input mRNAs (Brady et al., 1995 Curr Biol 5, 909-922.; Cheng et al., 1996 Proc Natl Acad Sci USA 93, 13158-63). This cDNA was then sequentially subtracted against cDNA from more mature CD34+CD38+ cells using biotinylated nucleotides incorporated into the subtractant population and avidin-mediated removal of common sequences.

Approximately 200 clones with increased expression in the G0 population were isolated and sequenced. Among these approximately one third were considered recombination artifact, one third were novel and those with homology to transmembrane molecules were considered for further evaluation. One clone encoded a seven transmembrane G-protein coupled receptor corresponding to a gene in GenBank (KIAA0001; accession number D13626 or NM_{—}014879, SEQ ID No.: 10) originally cloned from a CD34+ hematopoietic progenitor line, KG1, and subsequently identified in rat brain, but for which no function has been defined (Charlton et al., 1997 Brain Res 764, 141-8.; Nomura et al., 1994 [published erratum appears in DNA Res 1995 Aug. 31;2(4):following 210]. DNA Res 1, 27-35.). This clone was used to probe cDNA from unsubtracted G0 cells, sequentially subtracted G0 cells, and CD34+/CD38+ cells. The receptor was expressed in quiescent CD34+/CD38− cells, but not in CD34+/CD38+ cells (FIG. 1b).

cDNA from each round of subtractive hybridization probed with SC-GPR or GAPDH, demonstrated progressive enrichment of SC-GPR and depletion of GAPDH.

The stem cell-G protein-coupled receptor (SC-GPR) gene is predicted to encode a 338 amino acid protein and shows 20-30% similarity to known chemokine receptors. The sequence motif DRYYKIV (SEQ ID NO. 5), located at the end of transmembrane III showed similarity to the DRYLAIV (SEQ ID NO. 6) motif, which is a signature amino acid sequence for chemokine receptors (Youn et al., 1997 Blood 89, 4448-60.). The chromosomal location of SC-GPR was determined to coincide with the C-C chemokine receptor cluster on chromosome 3 (Napolitano et al., 1996 J Immunol 157, 2759-63.). These results suggest that SC-GPR is a member of the chemokine receptor family. Isolation and sequencing of the murine SC-GPR cDNA demonstrated high cross-species homology with human:mouse similarity of 90% and identity of 81% at the amino acid level.

SC-GPR is restricted in tissue expression, and its expression within hematopoietic cells is limited to primitive cells. Northern blots of human tissues demonstrated abundant signal in the heart, placenta and smooth muscle with minimal detectable signal in spleen, lymph node and thymus.

Further characterization of expression within hematopoietic cells was accomplished by using immunophenotypic populations sorted to high purity using FACS or, in the case of G0 cells, by the selected suicide strategy of CD34+ cells noted above. Probing cDNA from subpopulations of bone marrow derived stem or progenitor populations or fully mature blood cells, demonstrated highly restricted expression of SC-GPR to the 0 CD34+CD38− bone marrow cells. The indicated phenotype was assessed by poly-A primed RT-PCR and resulting cDNA probed with either SC-GPR or GAPDH sequences.

To further assess subpopulations of cells expressing SC-PGR1, we generated anti-peptide antiserum by immunization of New Zealand White rabbits with a peptide corresponding to the deduced sequence of first extracellular domain of SC-GPR. It was found that anti-SC-GPR recognizes SC-GPR and identifies a subset of CD34+CD38− fetal bone marrow cells. Reactivity of the antiserum to native protein was confirmed by immunoprecipitation of in vitro-translated HA-tagged SC-GPR protein using affinity purified antiserum followed by anti-HA western blot. HA-epitope-tagged SC-GPR cDNA was in vitro transcribed and translated. In vitro translated protein was immunoprecipitated with anti-HA tag monoclonal antibody and immunoblotted with an affinity purified SC-GPR polyclonal antibody. Similar results were obtained using anti-SC-GPR antibody for immunoprecipitation and anti-HA monoclonal antibody in subsequent immunoblotting.

The ability of the antiserum to selectively recognize SC-GPR expressed on the cell surface was demonstrated by flow cytometric analysis of 32D cells transfected with a retroviral expression construct (MSCV-GFP) of SC-GPR versus empty vector (control).

### Example 2

SC-GPR is Expressed on Primitive, Bone Marrow Localized Primary Hematopoietic Stem Cells.

Methods:

Immunocytochemistry

Immunocytochemistry was performed using avidin-biotin system and anti-HA mouse monoclonal antibody. All incubations were done at room temperature unless otherwise stated. Briefly, cells were fixed in 4% (v/v) paraformaldehyde for 20 min. Slides were incubated with anti-HA antibody (Babco, Richmond, Calif.) overnight at 40° C. followed by incubation with a biotinylated goat anti-mouse secondary antibody (Sigma, St. Louis, Mo.). Slides were then incubated with ExtrAvidin-FITC conjugate (Sigma). Slides were mounted in Fluoromount-G (Southern Biotechnology Associates, Inc. Birmingham, Ala.) and examined using fluorescence microscopy.

Results:

SC-GPR expression in primary hematopoietic populations was examined by flow cytometry. Immunomagnetic bead affinity purified CD34+ adult bone marrow cells were stained with epitope binding purified anti-SC-GPR and a second step fluorochrome, or, in independent experiments, directed conjugated FITC-anti-SC-GPR. SC-GPR expression was not evident on CD34 bright cells by standard FACS analysis while 1-2% of CD34 dim cells was positive. Reasoning that a very primitive population of cells may be in too low abundance in adult bone marrow to accurately detect by flow cytometry, we next evaluated human fetal bone marrow cells. Using this population, known to be enriched in hematopoietic stem cells, we observed 48+/−5% positivity in the CD34+CD38− cells in multiple independent samples. The composite data from 4 independent experiments is shown in Table 2 below.

TABLE 2

| Experiment | Positive [%] | Negative [%] |
|---|---|---|
| 1 | 59 | 41 |
| 2 | 42 | 58 |
| 3 | 40 | 60 |
| 4 | 51 | 49 |
| mean +/− SEM | 48 +/− 5.05 | 52 +/− 5.05 |

Of note, cells expressing SC-GPR were not detected in other fetal hematopoietic organs including the stem cell rich fetal liver, nor were they detected in umbilical cord blood also enriched for primitive cells. These data indicate a clear link of SC-GPR expression with bone marrow localization. Despite the link of CXCR-4 and integrins to bone marrow homing or retention, these molecules are expressed widely on hematopoietic cells including those in abundance in the circulation. This is not the case with SC-GPR, which has limited and specific expression.

### Example 3

SC-GPR is Associated with Cell Cycle Quiescence.

Results:

The CD34+CD38− subset of hematopoietic cells is regarded as a stem cell enriched population (Huang and Terstappen, 1994 Blood 83, 1515-26.; Terstappen et al., 1991 Blood 77, 1218-1221.). We subdivided a population of these cells from human fetal bone marrow based on expression of SC-GPR using cell sorting of immunostained cells. SC-GPR+CD34+CD38− and SC-GPR−CD34+CD38− subpopulations were then assayed for their cell cycle status. Staining the cells with Hoescht 33342 (Ho) was used to determine DNA content in order to distinguish between G1/G0 and G2-M+S phase, while the RNA dye, pyronin (PY), was used to distinguish G1 from G0 (Gothot et al., 1997 Blood 90, 4384-93).

Fetal bone marrow CD34+CD38− cells were predominantly in the Ho low fraction with only 2-3% in the G2-M+S phase with little difference noted between the SC-GPR+ and SC-GPR− populations. Among those cells in G0/G1 however, we noted markedly disproportionate fractions of cells in G1 versus G0 based on SC-GPR cell surface expression. Those cells not expressing SC-GPR− were predominantly in G1 (95%). Thus, CD34+CD38− cells expressing SC-GPR are disproportionately in G0 and are enriched for a stem cell functional phenotype. This contrasted markedly with SC-GPR+ cells in which only 57% were in G1 with the remainder in G0. Thus, the expression of SC-GPR preferentially occurs on a quiescent population of primitive bone marrow derived cells.

### Example 4

SC-GPR is Associated with Sustained Hematopoietic Cell Production in Long-Term Culture.

Methods:

CAFC/LTC-IC and CFC Assays

SC-GPR positive and negative cells were plated in triplicate in 1 ml methylcellulose media containing following recombinant human cytokines: SCF (50 ng/ml), GM-CSF (20 ng/ml), IL-3 (20 ng/ml), IL-6 (20 ng/ml), G-CSF (20 ng/ml) and EPO (3U/ml) (Methocult GF+ H4435, Stem Cell Technologies, Vancouver, Canada). Colonies were scored under an inverted microscope at 10 days after inoculation.

CAFC cultures were established according to described methods (Shen, H., et. al. (1999) J Virol 73, 728-37.; Sutherland, H. J., et. a (1990) Proc Natl Acad Sci USA 87, 3584-8.). Sorted cells were plated at 2-fold dilutions (3-6 dilutions/sample) on irradiated (15 Gy) primary human bone marrow stromal layers established at 33° C. and cultured in Human Long-term Bone Marrow Culture Media (Stem Cell Technology, Vancouver, Canada) at 37° C. Cultures were very gently re-fed with 50 μl medium after semidepletion weekly and the CAFCs and/or blast colonies were scored up to the 8th week. In some experiments, methycellulose was added to the well at 5 weeks and cultured for an additional 10 days prior to scoring LTC-IC by phase contrast microscopy. The absolute number of CAFCs was calculated using Poisson statistics.

Results:

SC-GPR+CD34+CD38− and SC-GPR−CD34+CD38− subpopulations were isolated by cell sorting from human fetal bone marrow and assayed for functional capacity by measuring colony forming cells (CFC) in methycellulose and long-term culture cobblestone area formation (CAFC) or long-term culture-initiating cell (LTC-IC) assays on bone marrow stroma. CFC assays measure more mature progenitors and CAFC or LTC-IC measure more primitive or stem-like cells. Among CD34+CD38− cells, SC-GPR+ cells performed poorly in the CFC assays compared with SC-GPR− cells (mean of 4.2 vs. 42.0 in 6 independent experiments, p=0.00003); CFC of SC-GPR+ versus SC-GPR− CD34+ CD38− cells (n=6, p=0.00003)). Producing few colonies could indicate either that SC-GPR+ cells are post-mitotic, terminally differentiated cells (unlikely in the CD34+ CD38− population) or are a more primitive, relatively cytokine unresponsive subset.

To discriminate between these, long-term assays were performed using limit dilutions of cells cultured on primary human marrow stroma. CAFC over time of SC-GPR+ versus SC-GPR− CD34+ CD38− cells calculated as the ratio relative to week 2. Cells were plated at 3-6 two-fold dilutions in replicate wells and scored each week. Three independent experiments scored weekly demonstrated low cobblestone area production with the SC-GPR+ cells. However, SC-GPR+ cells demonstrated a marked difference in capacity to provide a sustained output of hematopoietic colonies. In contrast to SC-GPR− cells, with which CAFC production declined over time as would be expected from a predominantly progenitor population (91% decline by week 8), SC-GPR+ cells consistently demonstrated sustained cobblestone formation over the same interval (47% decline). When the CAFC cultures were overlaid with cytokine supplemented methlycellulose at week 5 (LTC-IC assay), colonies emerged which were micropippetted and evaluated morphologically. Cells with myeloid cell histologic appearance were also observed. The morphology of cells derived from LTC-IC of SC-GPR+ CD34+ CD38− cells is consistent with mature myeloid lineage cells. Cells were isolated by micropippetting, stained with May-Grunwald-Giemsa and assessed by photo-light microscopy. Thus low level mature cell production was ongoing for prolonged intervals, characteristics consistent with a stem cell phenotype.

### Example 5

SC-GPR Activation and Ligand Source Identified.

Methods:

cDNA and Transfection

The SC-GPR coding region was generated by PCR using the human fetal thymus cDNA (Clontech, PaloAlto, Calif.) as the template and the primers SC-GPR-sBam and SC-GPR-aXho. The primer SC-GPR-sBam 5′-CGG GAT CCC GAA GTT ACA AGA TGA TCA ATT CAA CC (SEQ ID No.: 7) and SC-GPR-aXho 5′-CCG CTC GAG CGG AAG AGG GTA GGA ACT CA (SEQ ID No.: 8) correspond to position 346 and 1444 in the published sequence and span the entire coding region for SC-GPR of 338 amino acids. Products of expected size (bp) were cloned into the BamHI-XhoI polylinker sites of PcDNA3 (Invitrogen, Carsbad, Calif.). To create a HA tagged (YPYDVPDYA) (SEQ ID No.: 9) SC-GPR, untagged vector was used as a template for PCR. HA tag was inserted into either the N- or the C-terminus of a SC-GPR coding sequence. Sequence analysis of the final expression plasmids confirmed that there were no PCR generated mutations. Cos-7 cells were transfected with a HA-tagged KIA expression plasmid using Geneporter (GTS, San Diego, Calif.) according to the manufacturer's instructions. Retroviral transduction was performed as previously described using an MSCV-SC-GPR constructed by cloning the full length SC-GPR upstream of the IRES (Carlesso et al., 1999 Blood 93, 838-48.).

Intracellular Ca²⁺ Measurements

The calcium efflux assay was performed essentially as described (Shen et al., 1999 J Virol 73, 728-37). Cos-7 cells transfected with PcDNA 3 containing SC-GPR were cultured on glass slides and loaded with 5 uM fura-2/AM (Molecular Probes, Eugene, Oreg.) for 60 min at 37° C. in the dark. Cells were washed twice with PBS and once with DMEM. A slide was placed onto microscope stage (Nikon TE200) connected to a spectrofluorometer. The cells on the slide were submerged with loading buffer. Stroma-conditioned media from different hematopoietic organs were loaded onto the slide and fluorescence was measured. Data were presented as the relative ratio of fluorescence at an emission frequency of 510 nm and excitation frequencies of 340 nm and 380 nm.

Results:

We tested whether ligand binding to SC-GPR could result in a G protein mediated activation generating an intracellular calcium flux. We found that bone marrow conditioned medium induces a calcium flux in SC-GPR+ cells.

Cos-7 cells were transiently transfected with HA-epitope-tagged SC-GPR, or vector alone, and stained with anti-HA monoclonal antibody without cell permeabilization. The results demonstrated that SC-GPR was expressed on the cell surface of transduced cells.

Cells expressing SC-GPR were loaded with Fura-2 and monitored by fluorimetry following exposure to known chemokines or conditioned medium in an effort to define potential ligands. Intracellular calcium concentration was monitored by fluorescence ratio (F340/F380) plotted on the vertical axis. Similar results were obtained in three additional, independent experiments. SC-GPR transduced cells underwent calcium flux in response to selected conditioned media Screening of 13 known chemokines (IL-8, MIP1a, MIP1b, ATP, MCP 1, 3, 4, TARC, LTB4, RANTES, PAF, SDF-1 and PF4) failed to result in activation. However, conditioned medium from primary human fetal bone marrow stroma induced a calcium flux that was not observed when conditioned medium from other fetal hematopoietic orga Stroma sources was evaluated. Specifically spleen and thymic stroma conditioned medium failed to induce a calcium flux. Thus, SC-GPR was capable of inducing intracellular calcium shifts upon activation by a product restricted to bone marrow stroma. SC-GPR and a ligand source co-localize to the bone marrow.

### Example 6

SC-GPR Participates in Cell Localization.

Methods:

Chemotaxis Assay

Cell migration was assessed using 24-well chamber with 5-um pores (Corning Inc, NY). Human bone marrow stoma cells were cultured at confluence in the wells of lower compartment. The medium was changed 3-4 days before the assay. SC-GPR-MSCV or MSCV infected Jurkat cells (2×106 cells/ml) were placed in the upper wells of the chamber, respectively. The chamber was incubated at 37° C. in humidified air containing 5% CO₂, for 3 hours. After incubation, the filter was removed, and two independent investigators counted the number of migrated cells. All assays were done in triplicate.

In Vivo Homing Assay.

MSCV-SC-GPR and MSCV infected KG1 cells were labeled with carboxyfluroescein diacetate succinimidyl ester dye (CFDSE; Molecular Probes, Inc, Eugene, Oreg.) according to the manufacturer's instructions. Cells were resuspended in PBS at a concentration of 2×10⁶ml and then injected IV into lethally irradiated Balb/c mice. 22 hours after transplantation, cells were harvested from both the femur and tibia. The number of fluroscein-labeled cells in the entire sample was detected using a FACSCalibur.

Results:

To test whether SC-GPR activation induces cell migratory phenomena, transwell assays, and independently, Boyden chamber assays were performed. Adherent COS-7 cells will not function in these assays so we used non-adherent Jurkat cells and transduced them with SC-GPR in the MSCV retroviral vector. SC-GPR-MSCV Jurkat cells transmigrated toward a bone marrow stromal feeder layer significantly above the transmigration noted with control MSCV infected Jurkat cells (FIG. 6a). The results represent mean and s.e.m. of one of four independent experiments. Migration was expressed as a chemotactic index calculated from the percentage of cells in the test wells passing through a 5 μm filter over three hours, divided by the percentage of migration in media controls. Cells were counted by two independent investigators. The transmigration was less pronounced when we used conditioned medium from bone marrow (rather than a feeder layer), was minimally evident in response to thymic stroma conditioned media and not seen in response to conditioned media from spleen stroma Media alone also had no effect. Therefore, activation of SC-GPR by its putative ligand from bone marrow stroma mediated movement of transduced cells in vitro.

To define whether this stem cell restricted bone marrow specific chemokine receptor function to localize SC-GPR positive cells to the bone marrow environment, we performed in vivo studies on cell homing. These assays involved the use of the myeloid leukemic cell line, KG-1 infected with either a bicistronic retroviral vector encoding GFP alone or SC-GPR and GFP. We used KG-1 cells in this setting reasoning that SC-GPR may require other complementary molecules more likely to be on a primitive myeloid cell to fully manifest a marrow homing phenotype. Cells were sorted for GFP expression by flow cytometry and expanded. Cells were then stained with the cytoplasmic dye, CFSE, and injected into irradiated Balb/c mice 24 hours following radiation exposure. Animals were sacrificed after 12-18 hours and the bone marrow analyzed for the presence of CFSE positive cells. Those cells with a SC-GPR encoding retroviral vector were present in bone marrow in substantially greater abundance than cells transduced with control vector. Thus, SC-GPR transduced cells home to the bone marrow of irradiated mice. We performed flow cytometry of bone marrow from animals injected with untransduced KG-1 cells (control), empty vector transduced cells (MSCV) or SC-GPR-MSCV transduced cells (SC-GPR). Transduced cells were sorted for GFP+ prior to injection. Bone marrow was harvested 20-24 hours after injection and analyzed for CFDA-SE intensity.

Indirect mechanisms of altering cell homing by transduction of SC-GPR were evaluated. We examined the chemokine receptor, CXCR-4, expression by flow cytometry and noted no difference in cells infected with the control vector versus SC-GPR expressing vector. Since cell cycle status has been shown to alter integrins relevant for bone marrow homing (Becker et al., 1999 Exp Hematol 27, 533-41.), we evaluated infected cells and observed no difference in the proportion of KG-1 cells in S-G2-M between control or SC-GPR expressing cells. Therefore, the effect of SC-GPR on homing appears to be direct and correlates with in vitro observed transmigration.

### Example 7

Effect of SC-GPR on Primary Hematopoietic Cell Function.

Results:

KG-1 demonstrated no difference in cell cycling in liquid culture, however to assess primary cells, we transduced cord blood CD34+ cells with the SC-GPR or control retroviral vectors. The conditions for retroviral infection included pro-proliferative cytokines, IL-3, KL and FL. While these cytokines provide high transduction efficiencies, they also induce differentiation (Dao and Nolta, 1999). Leukemia 13, 1473-80.; Novelli et al., 1999 Hum Gene Ther 10, 2927-40.; Tsuji et al., 2000 Hum Gene Ther 11, 271-84). Cells were then plated in a bone marrow stromal feeder co-culture or in CFC assays. The ability of SC-GPR+ cells to generate CFC was significantly diminished compared with MSCV controls in four independent experiments (p=0.02) (total CFC were reduced with SC-GPR expression). These data correspond to the observations made in primary cells expressing SC-GPR where limited activity in mature cell (CFC) assays was noted. The absence of a ligand source, yet the presence of an effect suggests that primary cells may produce ligand in an autocrine manner, the receptor has constitutive activity when ectopically expressed or the presence of the receptor impedes other signaling events important for induction of the CFC phenotype. Thus, primary cord blood derived CD34+ cells alter their functional phenotype with ectopic SC-GPR expression.

In the presence of a bone marrow stroma feeder layer containing putative ligand, SC-GPR+ cells generated significantly higher numbers of CAFC than controls in three independent experiments (p=0.03)(CAFC are increased with SC-GPR expression in transduced primary CD34+ cells). These experiments were performed using limit dilution methods and analyzed by Poisson statistics. An additional LTC-IC experiment demonstrated LTC-IC only in the SC-GPR transduced population with no wells scoring positively in the vector alone transduced controls. Therefore, SC-GPR inhibits performance in a progenitor cell assay and enhances performance in stem cell assays. The basis for this could be simply inhibition of cell cycling, that CAFC are persistently present because of a delayed response to cytokines. Yet when we assayed DNA content by Hoescht 33342 analysis, we did not detect differences in the proportion of cells in G2-S in the SC-GPR+ population of transduced primary cells in liquid culture. Alternatively, SC-GPR is likely to induce or retain a more primitive phenotype, altering the differentiation program of primary cells.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

We claim:

1. A method for identifying a hematopoietic stem cell, comprising:
identifying the presence or absence of a stem cell G-protein coupled receptor (SC-GPR) on a putative hematopoietic stem cell, the presence of the SC-GPR being indicative of a hematopoietic stem cell.

2. The method of claim 1, wherein the presence of SC-GPR is determined by contacting the putative hematopoietic stem cell with a SC-GPR binding agent.

3. The method of claim 2, wherein the SC-GPR binding agent is a SC-GPR binding peptide.

4. The method of claim 3, wherein the SC-GPR binding peptide is selected from the group consisting of an anti-SC-GPR antibody and an anti-SC-GPR antibody fragment.

5. The method of claim 1, wherein the putative hematopoietic stem cell is present in a biological sample.

6. The method of claim 5, wherein the biological sample is a bone marrow sample.

7. The method of claim 5, wherein the biological sample is a blood sample.

8. The method of claim 1, wherein the presence of SC-GPR is determined by contacting the putative hematopoietic stem cell with a SC-GPR nucleic acid probe.

9. A method for isolating a hematopoietic stem cell, comprising:
contacting a sample containing a hematopoietic stem cell with a SC-GPR binding agent to isolate the hematopoietic stem cell from the sample, wherein the SC-GPR binding agent is not N-acetylglucosamine.

10. The method of claim 9, wherein the SC-GPR binding agent is a SC-GPR binding peptide.

11. The method of claim 10, wherein the SC-GPR binding peptide is an anti-SC-GPR antibody.

12. The method of claim 10, wherein the SC-GPR binding peptide is an anti-SC-GPR antibody fragment.

13. The method of claim 9, wherein the putative hematopoietic stem cell is present in a biological sample.

14. The method of claim 13, wherein the biological sample is a bone marrow sample.

15. The method of claim 13, wherein the biological sample is a blood sample.

16. The method of claim 9, wherein the hematopoietic stem cell is isolated using chromatography.

17. The method of claim 16, wherein the chromatography is column chromatography and the SC-GPR binding agent is fixed in the column.

18. The method of claim 9, wherein the hematopoietic stem cell is isolated using fluorescence activated cell sorting (FACS).

19. A kit, comprising:
a stem cell G-protein coupled receptor (SC-GPR) binding agent, and
instructions for contacting a hematopoietic stem cell with the SC-GPR binding agent to identify or isolate the hematopoietic stem cell.

20. The kit of claim 19, wherein the SC-GPR binding agent is a SC-GPR binding peptide.

21. The kit of claim 20, wherein the SC-GPR binding peptide is selected from the group consisting of an anti-SC-GPR antibody and an anti-SC-GPR antibody fragment.

22. The kit of claim 19, wherein the SC-GPR binding agent is fixed to a chromatography matrix.

23. A SC-GPR-transduced hematopoietic cell.

24. The cell of claim 23, wherein the SC-GPR-transduced hematopoietic cell is isolated from a bone marrow sample.

25. The cell of claim 23, wherein the SC-GPR-transduced hematopoietic cell is isolated from a blood sample.

26. The cell of claim 23, wherein the SC-GPR-transduced hematopoietic cell is prepared by transduction of a SC-GPR-negative hematopoietic cell with a nucleic acid of SEQ ID No. 10.

27. The cell of claim 23, further comprising an exogenous gene encoding a therapeutic agent.

28. A method for supplementing bone marrow, comprising:
administering to a subject a SC-GPR enriched hematopoietic cell population to supplement the bone marrow of the subject.

29. The method of claim 28, wherein the SC-GPR enriched hematopoietic cell population includes SC-GPR-transduced hematopoietic cells.

30. The method of claim 29, wherein the SC-GPR-transduced hematopoietic cells comprise SC-GPR-negative hematopoietic cells.

31. The method of claim 28, wherein the SC-GPR enriched hematopoietic cell population includes hematopoietic cells isolated by the method of claim B1.

32. The method of claim 29, wherein the SC-GPR enriched hematopoietic cell population includes hematopoietic cells isolated by the method of claim B1.

33. The method of claim 28, wherein the subject is a subject in need of a bone marrow transplant.

34. The method of claim 28, wherein the SC-GPR enriched hematopoietic cell population is administered to the peripheral blood of the subject.

35. A method for enhancing mobilization of hematopoietic stem cells, comprising:
administering to a subject a SC-GPR inhibitor to enhance mobilization of hematopoietic stem cells in the subject.

36. The method of claim 35, wherein the SC-GPR inhibitor is a SC-GPR antagonist.

37. The method of claim 35, wherein the SC-GPR inhibitor is a SC-GPR blocking agent.

38. The method of claim 35, wherein the SC-GPR inhibitor is a SC-GPR antisense molecule.

39. The method of claim 35, wherein the subject is a bone marrow donor.

40. A method for modulating hematopoietic cell function, comprising:
contacting a hematopoietic cell with a SC-GPR activator or a SC-GPR inhibitor to modulate hematopoietic cell function.

41. The method of claim 40, wherein the SC-GPR inhibitor is a SC-GPR antagonist.

42. The method of claim 40, wherein the SC-GPR inhibitor is a SC-GPR blocking agent.

43. The method of claim 40, wherein the SC-GPR inhibitor is a SC-GPR antisense agent.

44. The method of claim 40, wherein the SC-GPR activator is a SC-GPR nucleic acid.

45. The method of claim 40, wherein the SC-GPR activator is a SC-GPR agonist.

46. The method of claim 40, wherein the hematopoietic cell is not a hematopoietic stem cell.

47. A method for inducing hematopoietic stem cell quiescence, comprising:
contacting a hematopoietic stem cell with a SC-GPR activator to induce quiescence of the hematopoietic stem cell.

48. The method of claim 47, wherein the SC-GPR activator is a SC-GPR nucleic acid.

49. The method of claim 47, wherein the SC-GPR activator is a SC-GPR agonist.

50. The method of claim 47, wherein the contacting occurs in vitro.

51. A method for inhibiting hematopoietic stem cell-death, comprising:
inducing hematopoietic stem cell quiescence according to anyone of claims 47-50, to inhibit hematopoietic stem cell-death.

52. The method of claim 51, wherein the hematopoietic stem cell is under environmental stress.

53. A method for in vitro culture of hematopoietic cells to produce differentiated cells of non-hematopoietic lineage, comprising:
contacting a hematopoietic cell with a SC-GPR molecule under conditions sufficient to confer hematopoietic stem cell characteristics to the hematopoietic cell, and
culturing the hematopoietic cell having stem cell characteristics in an environment that promotes hematopoietic cell differentiation, under conditions and for a period of time to produce differentiated cells of non-hematopoietic lineage.


SEQUENCE LISTING (10 sequences)

SEQ ID NO: 1 - protein, 338 aa, Homo sapiens
MINSTSTQPPDESCSQNLLITQQIIPVLYCMVFIAGILLNGVSGWIFFYVPSSKSFIIYLKNIVIADFVMSLTFPFKILGDSGLGPWQLNVFVCRVSAVLFYVNMYVSIVFFGLISFDRYYKIVKPLWTSFIQSVSYSKLLSVIVWMLMLLLAVPNIILTNQSVREVTQIKCIELKSELGRKWHKASNYIFVAIFWIVFLLLIVFYTAITKKIFKSHLKSSRNSTSVKKKSSRNIFSIVFVFFVCFVPYHIARIPYTKSQTEAHYSCQSKEILRYMKEFTLLLSAANVCLDPIIYFFLCQPFREILCKKLHIPLKAQNDLDISRIKRGNTTLESTDTL

SEQ ID NO: 2 - protein, 338 aa, Mus musculus
MNNSTTTDPPNQPCSWNTLITKQIIPVLYGMVFITGLLLNGISGWIFFYVPSSKSFIIYLKNIVVADFLMGLTFPFKVLGDSGLGPWQVNVFVCRVSAVIFYVNMYVSIVFFGLISFDRYYKIVKPLLTSIVQSVNYSKLLSVLVWMLMLLLAVPNIILTNQGVKEVTKIQCMELKNELGRKWHKASNYIFVSIFWVVFLLLIVFYTAITRKIFKSHLKSRKNSTSVKRKSSRNIFSIVLVFVVCFVPYHIARIPYTKSQTEGHYSCRTKETLLYAKEFTLLLSAANVCLDPIIYFFLCQPFREVLNKKLHMSLKVQNDLEVSKTKRENAIHESTDTL

SEQ ID NO: 3 - protein, 305 aa, Rattus norvegicus
MDNTTTTEPPKQPCTRNTLITQQIIPMLYCVVFITGVLLNGISGWIFFYVPSSKSFIIYLKNIVVADFLMGLTFPFKVLSDSGLGPWQLNVFVFRVSAVIFYVNMYVSIAFFGLISFDRYYKIVKPLLVSIVQSVNYSKVLSVLVWVLMLLLAVPNIILTNQSVKDVTNIQCMELKNELGRKWHKASNYVFVSIFWIVFLLLTVFYMAITRKIFKSHLKSRKNSISVKRKSSRNIFSIVLAFVACFAPYHVARIPYTKSQTEGHYSCQAKETLLYTKEFTLLLSAANVCLDPISISSYASRLEKS

SEQ ID NO: 4 - protein, 17 aa, Homo sapiens
  PEPTIDE (1)...(17): N-SC-GPR
MINSTSTQPPDESCSQN

SEQ ID NO: 5 - protein, 7 aa, Homo sapiens
  DOMAIN (1)...(7)
DRYYKIV

SEQ ID NO: 6 - protein, 7 aa, Homo sapiens
  DOMAIN (1)...(7)
DRYLAIV

SEQ ID NO: 7 - DNA, 35 bp, Homo sapiens
  primer SC-GPR-sBam
cgggatcccgaagttacaagatgatcaattcaacc

SEQ ID NO: 8 - DNA, 29 bp, Homo sapiens
  primer SC-GPR-aXho
ccgctcgagcggaagagggtaggaactca

SEQ ID NO: 9 - protein, 9 aa, Artificial Sequence
  HA-tag
YPYDVPDYA

SEQ ID NO: 10 - DNA, 2416 bp, Homo sapiens
  CDS (217)...(1230)
gaacagtgttaccttggagcctacaatgagaggtatttcaaaatgagtgaagcatgactctcacagatgaaggcctagacgcaggatctttaatggaaaaacacttgggccacttcaagacgacaaacgctcactgggcaaaacaccttcactgaaaagagacctcatattatgcaaaaaaaatcttaagaggcctctgccttcagaagttacaagatgatcaattcaacctccacacagcctccagatgaatcctgctctcagaacctcctgatcactcagcagatcattcctgtgctgtactgtatggtcttcattgcgggaatcctactcaatggagtgtcaggatggatattcttttacgtgcccagctctaagagtttcatcatctatctcaagaacattgttattgctgactttgtgatgagcctgacttttcctttcaagatccttggtgactcaggccttggtccctggcagctgaacgtgtttgtgtgcagggtctctgccgtgctcttctacgtcaacatgtacgtcagcattgtgttctttgggctcatcagctttgacaggtattataaaattgtaaagcctctttggacttctttcatccagtcagtgagttacagcaaacttctgtcagtgatagtatggatgctcatgctcctccttgctgttccaaatattattctcaccaaccagagtgttagggaggttacacaaataaaatgtatagaactgaaaagtgaactgggacggaagtggcacaaagcatcaaactacatcttcgtggccatcttctggattgtgtttcttttgttaatcgttttctatactgctatcacaaagaaaatctttaagtcccaccttaagtcaagtcggaattccacttcggtcaaaaagaaatctagccgcaacatattcagcatcgtgtttgtgttttttgtctgttttgtaccttaccatattgccagaatcccctacacaaagagtcagaccgaagctcattacagctgccagtcaaaagaaatcttgcggtatatgaaagaattcactctgctactatctgctgcaaatgtatgcttggaccctattatttatttctttctatgccagccgtttagggaaatcttatgtaagaaattgcacattccattaaaagctcagaatgacctagacatttccagaatcaaaagaggaaatacaacacttgaaagcacagatactttgtgagttcctaccctcttccaaagaaagaccacgtgtgcatgttgtcatcttcaattacataacagaaatcaataagatatgtgccctcatcataaatatcatctctagcactgccatccaatttagttcaataaaattcaaatataagtttccatgcttttttgtaacatcaaagaaaacatacccatcagtaatttctctaatactgacctttctattctctattaataaaaaattaatacatacaattattcaattctattatattaaaataagttaaagtttataaccactagtctggtcagttaatgtagaaatttaaatagtaaataaaacacaacataatcaaagacaactcactcaggcatcttctttctctaaataccagaatctagtatgtaattgttttcaacactgtccttaaagactaacttgaaagcaggcacagtttgatgaagggctagagagctgtttgcaataaaaagtcaggtttttttcctgatttgaagaagcaggaaaagctgacacccagacaatcacttaagaaaccccttattgatgtatttcatggcactgcaaaggaagaggaatattaattgtatacttagcaagaaaattttttttttctgatagcactttgaggatattagatacatgctaaatatgttttctacaaagacttacgtcatttaatgagcctggggttctggtgttagaatatttttaagtaggctttactgagagaaactaaatattggcatacgttatcagcaacttcccctgttcaatagtatgggaaaaataagatgactgggaaaaagacacacccacaccgtagaacatatattaatctactggcgaatgggaaaggagaccattttcttagaaagcaaataaacttgatttttttaaatctaaaatttacattaatgagtgcaaaataacacataaaatgaaaattcacacatcacatttttctggaaaacagacggattttacttctggagacatggcatacggttactgacttatgagctaccaaaactaaattctttctctgctattaactggctagaagacattcatctatttttcaaatgttctttcaaaacatttttataagtaatgtttgtatctatttcatgctttactgtctatatactaataaagaaatgttttaatactg